(19) United States
(12) Patent Application Publication
(10) Pub. No.: US 2022/0310196 A1
(43) Pub. Date: Sep. 29, 2022

(54) SYNTHETIC BIOLOGICAL CHARACTERISTIC GENERATOR BASED ON REAL BIOLOGICAL DATA SIGNATURES

(71) Applicant: INSILICO MEDICINE IP LIMITED, Hong Kong (HK)

(72) Inventors: Daniil POLYKOVSKIY, Moscow (RU); Daniil KORBUT, Moscow (RU); Aleksandrs ZAVORONKOVS, Pak Shek Kok (HK)

(21) Appl. No.: 17/596,888

(22) PCT Filed: Jun. 20, 2020
(86) PCT No.: PCT/IB2020/055827

§ 371 Date: Dec. 20, 2021

Related U.S. Application Data
(60) Provisional application No. 62/864,334, filed on Jun. 20, 2019.

(51) Int. Cl. G16B 5/00; G16B 20/00; G16B 40/20

(52) U.S. Cl. CPC G16B 5/00; G16B 20/00; G16B 40/20

(57) ABSTRACT

Creating synthetic biological data for a subject can include: (a) receiving a real biological data signature derived from a biological sample of the subject; (b) creating input vectors based on the real biological data signature; (c) inputting the input vectors into a machine learning platform; (d) generating a predicted biological data signature of the subject based on the input vectors, wherein the predicted biological data signature includes synthetic biological data specific to the subject; and (e) preparing a report that includes the synthetic biological data of the subject. Biological pathway activation signatures can be genomics, transcriptomics, proteomics, metabolomics, lipidomics, glycomics, methylomics, or secretomics. Conditioning latent codes of the input vectors in a latent space of the machine learning platform with at least one constraint of an attribute of the subject is performed so the predicted biological data signature is based on the at least one constraint.

### CROSS-REFERENCE

This patent application claims priority to U.S. Provisional Application No. 62/864,334 filed Jun. 20, 2019, which application is incorporated herein by specific reference in its entirety.

### BACKGROUND

With the major demographic shift towards the elderly population, there a growing need for interventions that able to extend lifespan. At the same time, such interventions may require measures of biological aging at the individual level, which can quantify age acceleration and deceleration.

While aging may be a complex multifactorial process with no single cause or treatment, the issue of whether aging can be classified as the disease is widely debated. An animal's survival strongly depends on its ability to maintain homeostasis, achieved partly through intracellular and intercellular communication within and among different tissues. The physiological activity that maintains the homeostasis provides a biological data signature for different cells, tissues, organs, or the entire animal organism. This biological data signature can be obtained from biological samples of the animal by standard biotechnological protocols. The biological data signature can be used for assessing the health of the animal as well as determining the biological age of the animal. The biological age may be different from the chronological age, and thereby provide information for health, disease potential, and deviation from the chronological age (e.g., premature aging).

At least two general concepts of age exist in the art. One, “chronological age” is simply the actual calendar time an organism or human has been alive. Another one, called “biological age” or “physiological age”, which is a particular focus of the present invention, is related to the physiological health of the individual, and biomarkers thereof, whether transcriptomic or proteomic or other biological data signature. Biological age is associated with how well organs and regulatory systems of the body are performing and at what extent the general homeostasis at all levels of the organism is being maintained, as such functions generally decline with time and age.

It is known that the lifespan of different cells and tissues varies substantially. Although aging affects gene expression and protein production as well as other biological signatures differently in different tissues, the biological signature (e.g., genomics) is highly tissue specific and depends on functions in the tissue, such as by the proteins produced as the final product of gene expression. As the regeneration rates and associated with it gene expression and protein production patterns vary, external effectors, such as small molecules, have different effect on different tissues. As a result, gene expression and protein production can provide specific signatures for the cells, tissue, organs, body fluids, or organism that can be studied to find information for interventions that could bring the tissues, organ, or organism (e.g., person) back to a younger state of biological age without an additional adverse effects on other tissues.

The measurement of any physiological process of an organism is typically done with a set of predefined biomarkers. A biomarker can be defined as a characteristic that is objectively measured and evaluated as an indicator of normal biological processes, pathogenic processes, or pharmacologic responses to a therapeutic intervention. Biomarkers are chosen by scientists in order to measure a very-well defined process within the body.

A biomarker is a characteristic that is objectively measured and evaluated as an indicator of normal biological processes, pathogenic processes, or pharmacologic responses to a therapeutic intervention. An aging clock is a model that predicts the biological age of an individual based on a set of biomarkers. In a sense, it can be treated as a standalone composite biomarker. According to the American Federation for Aging Research (AFAR), a biomarker should satisfy the following conditions to be regarded as a biomarker of aging: 1) It is a better predictor of mortality than chronological age; 2) It predicts aging rates; 3) It is responsive to aging—not diseases; 4) Can be applied to both humans and model organisms; and 5) It can be tested repeatedly.

Many biomarkers of aging have been proposed including telomere length, intracellular and extracellular aggregates, racemization of the amino acids and genetic instability. Gene expression and DNA methylation profiles change during aging, which also may be used as biomarkers of aging. As a result, protein production profiles that are translated from the genetically expressed mRNA may correspondingly be used as biomarkers of aging. Many studies analyzing transcriptomes or proteomes of biopsies in a variety of diseases indicated that age and sex of the patient have significant effects on gene expression and subsequent protein production and that there are noticeable changes in gene expression with age in mice, resulting in development of mouse aging gene expression databases and in humans.

Advances in the generation of biological and medical data have resulted in the development of multiple new types of aging biomarkers including epigenetic clocks (Hannum et al., 2013; Horvath, 2013), transcriptomic clocks (Peters et al., 2015). And while all of those models were developed with conventional shallow machine learning approaches mainly using regularized linear regression those results suggest that gradual changes during aging can be tracked using various data types, including transcriptome, with reasonable accuracy.

With the advent of graphic processing computing deep learning revolutionized many areas including biomedicine (Mamoshina et al., 2016). First published in 2016, predictors of chronological and biological age developed using deep learning (DL) are rapidly gaining popularity in the aging research community. Multiple deep-learning-based aging clocks have been published including hematological (Mamoshina et al., 2018a, 2019; Putin et al., 2016), facial (Bobrov et al., 2018), transcriptomic (Mamoshina et al., 2018b), microbiomic (Galkin et al., 2020).

A common strategy to study changes associated with aging is to build a regression model that receives a vector of patient profile values such as gene expression levels or protein levels and outputs a continuous value of patient age. At the same time, identification of the prognostic markers of ageing remains a challenge.

Previously, studies have utilized biological data signatures obtained from biological samples for the animal. However, it may not always be possible to obtain a physical biological sample and obtain the corresponding biological data profile. Therefore, it may be advantageous to be able to obtain biological data that is not directly from a biological sample.

### SUMMARY

In some embodiments, a method of creating synthetic biological data for a subject can include: (a) receiving a real biological data signature derived from a biological sample of the subject; (b) creating input vectors based on the real biological data signature; (c) inputting the input vectors into a machine learning platform; (d) generating a predicted biological data signature of the subject based on the input vectors by the machine learning platform, wherein the predicted biological data signature includes synthetic biological data specific to the subject; and (e) preparing a report that includes the synthetic biological data of the subject. In some aspects, the real biological data signature is based on biological pathway activation signatures for genomics, transcriptomics, proteomics, metabolomics, lipidomics, glycomics, methylomics, or secretomics, and the predicted biological data corresponds with the biological activation signature.

In some embodiments, the methods can include: comparing the predicted biological data signature with the real biological data signature of the subject; determining a difference between the synthetic biological data of the subject with the real biological sample of the subject; and preparing the report with that identifies difference between the synthetic biological data with the real biological sample of the subject.

In some embodiments, the method includes conditioning latent codes of the input vectors in a latent space of the machine learning platform with at least one constraint of an attribute of the subject, such that the predicted biological data signature is based on the at least one constraint. In some aspects, the predicted biological data signature is generated based on at least one attribute of the subject, wherein the attribute is selected from age, sex, tissue types, ethnicity, life expectancy, or combination thereof of the subject.

In some embodiments, the synthetic biological data is for a defined biological age of the subject, wherein the predicted biological data signature represents a biological data signature of the subject at the defined biological age. In some aspects, the synthetic biological data is for one of: an aging simulation to increase a biological age of the biological data signature of the subject; or a rejuvenation simulation to decrease a biological age of the biological data signature of the subject.

In some embodiments, a received real biological data signature is compared with the generated predicted biological data signature to identify at least one biological pathway that is useful for predicting at least one of: age, sex, tissue types, cell types, ethnicity, life expectancy, and combinations thereof. In some aspects, the machine learning platform predicts a biological age, sex, tissue types, cell types, ethnicity, life expectancy or combinations thereof of the synthetic biological data.

In some embodiments, a computer program product comprising a tangible, non-transitory computer readable medium having a computer readable program code stored thereon, the code being executable by a processor to perform the methods described herein.

In some embodiment, a computing system having the computer program product can be used to perform the methods described herein.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A shows a schematic of a protocol for using a generative model to produce synthetic transcriptome profiles given age, sex, race and batch ID of the dataset that is based from a real biological sample.

FIG. 1B includes a flow chart for generating synthetic biological data from measured biological data with a generative model.

FIG. 1C shows a schematic diagram of a generative model used to produce synthetic transcriptome profiles.

FIG. 2 illustrates the personalized transcriptional vectors of the latent space for the generative model and the transformation of the real data (top) given the ID of the dataset to eliminate batch effect in the synthetic transcriptional vectors (bottom).

FIG. 3 illustrates the dependency of age consistency loss from the difference between actual chronological age and target age.

FIG. 4 shows the clustering of NETO2 gene age trajectories shows four categories of expression profiles.

FIGS. 5A-5E shows graphs of clustering of ALOX5 gene age trajectories (FIG. 5A) from four categories of expression profiles (FIGS. 5B-5E).

FIG. 6A shows a biological data profile with the top 9 signaling pathways perturbed by the aging protocol, where upregulated genes (pathways) are shown in red and the down-regulated genes (pathways) are shown in green, an where the saturation color denotes the perturbation amplitude: 401—Integrin-linked kinase signature; 402—Rapid glucocorticoid signature; 403—Thromboxane A2 receptor signature; 404—Signaling events mediated by VEGFR1 and VEGFR2; 405—Aurora B signature; 406—Glypican 2 network pathway signature; 407—PAR4 mediated thrombin signature; 408—Plasma membrane estrogen receptor signature; and 409—CXCR3 mediated signature.

FIG. 6B shows a biological data profile with the top 9 signaling pathways perturbed by the rejuvenation protocol (e.g., reverse to reduce age or “de-aging”), where upregulated genes (pathways) are shown in red and the down-regulated genes (pathways) are shown in green, an where the saturation color denotes the perturbation amplitude: 421—Aurora B signature; 422—Rapid glucocorticoid signature; 423—Thromboxane A2 receptor signature; 424—PAR4 mediated thrombin signature; 425—CXCR3 mediated signature; 426—Signaling event mediated by HDAC Class II; 427—Signaling events mediated by VEGFR1 and VEGFR2; 428—Visual signal transduction; and 429—IL8 and CXCR2 mediated signature.

FIG. 7 illustrates an embodiment of a computing system that can perform the computing methods described herein.

The elements in the figures are arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention relates to biomarkers of human biological aging. In some aspects, the invention relates to biomarkers based on gene expression, also called transcriptomic data, which provide metrics and estimates of the biological age of organisms, including humans. However, the biomarkers can be other omic biomarkers as recited herein, and the biological data can include an omics signature of biological data. For example, the omics signature is genomics, transcriptomics, proteomics, metabolomics, lipidomics, glycomics, methylomics, or secretomics. While transcriptomic biomarkers and biological data are described herein, the discussion is also applicable to the other omic biomarkers and data. An omic prognostic aging marker is provided based on such biomarkers and use thereof. For example, methods can include: obtaining the biological sample from the subject; and obtaining the real biological data signature by performing a measurement of the genomics, transcriptomics, proteomics, metabolomics, lipidomics, glycomics, methylomics, or secretomics.

Additionally, machine learning and deep learning techniques are utilized to assess the transcriptomic data and/or proteomic data and/or other omic data and the biomarkers of human biological aging. The invention provides methods that can be utilized to assess the course of transcriptome biological aging (e.g., computer methods performed on transcriptomic data of a subject), and then treat biological aging (e.g., therapeutic methods performed on subject). The invention includes methods, system, apparatus, computer program product, among others, to carry out the following protocols, such as for generating a predicted biological data signature for a subject based on the real biological data signature for the subject. The predicted biological data signature can be based on a perturbation or setting of at least one attribute of the subject for the synthetic data signature. The predicted biological data signature can be based on a simulation by a computer program for biological pathway activation signatures for genomics, transcriptomics, proteomics, metabolomics, lipidomics, glycomics, methylomics, or secretomics.

In some embodiments, the predicted biological data signature is generated based on at least one attribute of the subject, wherein the attribute is selected from age, sex, tissue types, ethnicity, life expectancy, or combination thereof of the subject. In some aspects, a parameter of one of these attributes can be set (e.g., age 65) to provide the predicted biological data for that defined attribute.

In some embodiments, a method of creating a prognostic aging marker is provided. The method can include receiving a biological data signature (e.g., transcriptome signature) derived from patient tissue or organ or the like, which can be obtained by processing a biological sample to determine the biological data signature, such as biomarkers signatures. Based on the biological data signature, the method can include providing input vectors to a machine learning platform. The machine learning platform processes the input vectors in order to generate output that includes a generated biological data signature given an age or desired age. In some aspects, the generated biological data signature is specific to the tissue, fluid, cell, or organ, or specific to a characteristic of the tissue, fluid cell, or organ. In some aspects, the method can include repeating one or more of the steps (e.g., receiving biological data signature and/or inputting the input vectors and/or generating output) for determining or creating a second generated biological data signature, such as for the same subject, cell, organ or tissue, or a different subject, cell, organ or tissue. In some aspects, the two prognostic aging markers are combined to create a synthetic prognostic marker that addresses biological aging at the tissue, organ, fluid, cell, or organism level for the subject or more than one subject. In some aspects, the method can include repeating one or more of the steps a plurality of times to create a plurality prognostic aging markers, such as for two or more sources of a biological sample in a subject, or for two or more subjects. In some aspects, the transcriptome signature and/or input vectors and/or generated output which is derived from a non-senescent tissue or organ of the patient or another organism.

In some embodiments, a subset of the biomarkers (e.g., genes or gene sets) of generated biological data (e.g., transcriptional) signature is selected as targets for anti-aging therapies. This can be based on the biological data signature and/or generated biological data signature output. In some aspects, a biological marker can provide a biological pathway or related subset of the genes or gene sets that can be selected as targets for aging rejuvenating therapies, where targets can be subsets of the proteins or protein sets that correspond with the selected biological pathway or subset of the genes or gene sets. In some aspects, a subset of genes or gene sets is selected as targets for personalized rejuvenating therapies using generated signature with a desired age of the patient.

In some embodiments, the biological data includes transcriptome signatures are based on signaling pathway activation signatures. In some aspects, the input transcriptome signatures profiles are derived from a microarray platform. In some aspects, the input transcriptome signatures profiles are derived from an RNA sequencing platform. In some aspects, the input transcriptome signatures profiles are derived from a quantitative reverse transcription polymerase chain reaction. In some aspects, the input transcriptome signature profiles are derived from a computer model for simulating gene expression data. In some aspects, the transcriptional signature is specific to a tissue or organ, or specific to a characteristic of the tissue or organ. The various omic biological data can obtain the biomarkers thereof by known methods.

In some embodiments, a method of creating synthetic data for a subject can include: receiving a transcriptome signature derived from patient same; providing input vectors to a machine learning platform; and generating synthetic sample with characteristics of the patient. The steps can be repeated to create additional synthetic data for a single subject or for a plurality of synthetic data a plurality of subjects. The synthetic data can be specific from the type of sample, such as tissue, organ, fluid, cells, or other. The synthetic data can provide a characteristic of the biology of the subject. The synthetic sample can be generated based on a defined or given age, sex, tissue types, ethnicity, life expectancy, or combination thereof of the subject. The characteristics of the synthetic sample can be predicted by the machine learning platform, for any of the age, sex, tissue types, ethnicity, life expectancy, or combination thereof of the subject. The given age, sex, tissue types, ethnicity, life expectancy, or combination thereof of the subject may be changed or specified to determine variations of synthetic biological data based on the changes or specificity. For example, a subject being a chronological age of 45 can have an aging acceleration to a defined biological age (e.g., 60) to obtain a predicted synthetic biological sample under this constraint, or an aging rejuvenation to a defined biological age (e.g., 30) to obtain a predicted synthetic biological sample as a target for rejuvenation purposes. Comparing the real biological data signature with the predicted biological data signature can provide indications of the biomarkers that can be important for assessing health or a biological age with regard to age, sex, tissue types, cell types, ethnicity, life expectancy prediction, and combinations thereof.

In some embodiments, the machine learning platform comprises one or more deep neural networks. In some aspects, the machine learning platform comprises one or generative adversarial networks. In some aspects, the machine learning platform comprises an adversarial autoencoder architecture. In some aspects, the machine learning platform comprises a feature importance analysis for ranking biomarkers, such as genes or gene sets, by their importance in age prediction.

In some embodiments, the machine learning platform can be configured for performing a biological signal activation analysis with the synthetic biological data and determining a health status of the subject. For example, the health status can be a predicted future health status of the subject. As described herein, the health status can be used for identifying a therapeutic protocol to improve the predicted future health status of the subject. In some aspects, the health status of the subject is an aging rate of the subject. In some aspects, the method can include tracking the aging rate of the subject over a time period.

In some embodiments, the machine learning platform can process a synthetic sample and then make a prediction for a synthetic biological data signature for an age, sex, tissue types, cell types, ethnicity, life expectancy prediction, and combinations thereof. Also, the machine learning platform can process the synthetic sample to predict an attribute of the subject, such as the age, sex, tissue types, cell types, ethnicity, life expectancy prediction, and combinations thereof.

In some embodiments, the machine learning platform includes a feature importance analysis module for ranking biomarkers by their importance in age prediction. The feature importance analysis can also be used for ranking the biomarkers by their importance in sex prediction. Additionally, the feature importance analysis for ranking the biomarkers by their importance in age pathology prediction. Also, the biomarker signatures that are real and synthetic can be correlated with the subject that provides the biological sample. As such, the biomarker signatures and associated pathways that are real and synthetic can be correlated with actual age, sex, ethnicity or life expectancy of the subject. Corelating the biomarker signatures that are real and synthetic can be used for a prognosis of life expectancy and probability of survival before, during or after an intervention or therapy. Accordingly, the method can include performing feature importance analysis for ranking biological data by importance in age prediction by using the real biological data signature, and identifying a subset biological markers of the biological pathway activation signature thereof that are selected as indicators of a condition of the subject. In some aspects, the method can include identifying at least one biological target associated with the condition, wherein modulation of the at least one biological target modulates at least one biomarker of the identified subset of biological markers.

In some embodiments, a method of creating synthetic biological data for a subject can include: (a) receiving a real biological data signature derived from a biological sample of the subject; (b) creating input vectors based on the real biological data signature; (c) inputting the input vectors into a machine learning platform; (d) generating a predicted biological data signature of the subject based on the input vectors by the machine learning platform, wherein the predicted biological data signature includes synthetic biological data specific to the subject; and (e) preparing a report that includes the synthetic biological data of the subject. In some aspects, the methods can include creating at least a second biological data signature by repeating any one or more of steps (a), (b), (c), and/or (d), wherein the second biological data signature is based on a second real biological data signature from the biological sample of the subject, a different biological sample of the subject, or a second biological sample of a second subject. Optionally, a report can be prepared that includes a second synthetic biological data of the second biological data signature.

In some embodiments, the methods can include: comparing the predicted biological data signature with the real biological data signature of the subject; determining a difference between the synthetic biological data of the subject with the real biological sample of the subject; and preparing the report with that identifies difference between the synthetic biological data with the real biological sample of the subject. In some aspects, the method can include identification of at least one biomarker having a difference between the synthetic biological data with the real biological sample of the subject. In some aspects, the method can include identifying at least one biological target, wherein modulation of the at least one biological target modulates the identified at least one biomarker.

In some embodiments, after a defined time period, the method can include performing steps (a), (b), (c), (d), and (e) in a second iteration; comparing the initial report with the report of the second iteration; and determining a change in the predicted biological data signature over the defined time period. The defined time period may also include a treatment or therapeutic regimen or lifestyle change. Then, the method can include determining whether the treatment, therapeutic regimen or lifestyle changed the predicted biological data signature. If it hanged the predicted biological data signature, then determine whether or not to: continue therapeutic regimen, change therapeutic regimen, or stop therapeutic regimen. If it does not change the predicted biological data signature, then determine whether or not to: continue therapeutic regimen, change therapeutic regimen, or stop therapeutic regimen. In some aspects, the methods can include identification of at least one biomarker having a difference over the defined time period. In some aspects, the methods can include identifying at least one biological target, wherein modulation of the at least one biological target modulates the identified at least one biomarker. In some aspects, the methods can include determining an aging rate over the defined time period based on the change in the predicted biological data signature; and tracking the change in the predicted biological data signature over the defined time period.

In some embodiments, the real biological data signature is based on biological pathway activation signatures for genomics, transcriptomics, proteomics, metabolomics, lipidomics, glycomics, methylomics, or secretomics, and the predicted biological data corresponds with the biological activation signature. In some aspects, the methods can include: correlating a genomics profile with the predicted biological data signature of the subject; correlating a proteomics profile with the predicted biological data signature of the subject; correlating a transcriptomics profile with the predicted biological data signature of the subject; correlating a metabolomics profile with the predicted biological data signature of the subject; correlating a lipidomics profile with the predicted biological data signature of the subject; correlating a glycomics profile with the predicted biological data signature of the subject; correlating a secretomics profile with the predicted biological data signature of the subject; or correlating a methylomics profile with the predicted biological data signature of the subject. The methods can also include correlating the predicted biological data signature with a predicted biological age of the subject.

In some embodiments, the synthetic biological data is for a defined biological age of the subject, wherein the predicted biological data signature represents a biological data signature of the subject at the defined biological age. This can allow for predicting the health of a subject at some time in the future. Alternatively, this can allow for predicting what the health of the subject could be if they were living a healthier lifestyle or activity trying to treat or overcome an adverse health condition. The synthetic biological data can be for one of: an aging simulation to increase a biological age of the biological data signature of the subject; or a rejuvenation simulation to decrease a biological age of the biological data signature of the subject. In some aspects, the methods can include identification of at least one biomarker having a difference between the real biological sample of the subject with the biological data signature of the aging simulation or the rejuvenation simulation. In some aspects, the methods can include identifying at least one biological target, wherein modulation of the at least one biological target modulates the identified at least one biomarker.

In some embodiments, the received real biological data signature is compared with the generated predicted biological data signature to identify at least one biological pathway that is useful for predicting at least one of: age, sex, tissue types, cell types, ethnicity, life expectancy, and combinations thereof. The machine learning platform can predict a biological age, sex, tissue types, cell types, ethnicity, life expectancy or combinations thereof of the synthetic biological data.

In some embodiments, the method can include comparing a generated synthetic biological data profile of an individual with an actual biological data profile of the individual. In some aspects, the method can include correlating gene expression levels with gene expression levels of a generated transcriptional signature.

In some embodiments, the method can include comparing a generated biological data profile of an individual with an actual biological data profile of the individual, wherein the comparison further comprises a prognosis of the life expectancy. In some aspects, the method can include comparing a generated biological data profile of an individual with an actual biological data profile of the individual, wherein the compassion further includes the generation of a signaling pathway signature. In some aspects, the method can include comparing a generated biological data profile of an individual with an actual biological data profile of the individual, wherein the comparison further comprises a prognosis of the life expectancy and probability of survival of the patient during treatment. In some aspects, the method can include comparing a generated biological data profile of an individual with an actual biological data profile of the individual, wherein the comparison comprises an outcome measure of the efficacy of the therapies. In some aspects, the method can include comparing a generated biological data profile of an individual with an actual biological data profile of the individual, wherein the comparison comprises an outcome measure probability of a patient developing adverse reactions to the therapies. In some aspects, the method can include comparing a generated biological data profile of an individual with an actual biological data profile of the individual, wherein the comparison comprises an optimal therapy.

In some embodiments, a method can include developing an intervention based on the output. In some embodiments, a method can include developing a medical therapy based on the output. In some aspects, a method can include developing a senolytic therapy based on the generated output. In some aspects, a method can include developing a senoremdiation therapy based on the generated output. In some aspects, a method can include developing a therapy that combines multiple interventions based on the generated output.

In part, because the method includes one or more prognostic biomarkers of aging, it could be used to track the efficacy of the anti-aging therapies, such as senolytic therapy and senoremdiation therapies. The method can be used to generate a biological data (e.g., transcriptional) signature given the desired and this biological data signature can be compared with a current biological data signature to identify the changes that need to be done to biological data signature to decrease its aging levels (e.g., make transcriptome younger or increase life expectancy of the patient, etc.).

The proposed method can be combed with biological aging clocks to predict age of generated biological data signatures.

The invention also includes methods for creating a prognostic aging marker for a patient, the method comprising: (a) receiving a first biological data signature derived from patient tissue or organ; (b) computing the generated biological data signature; (c) calculate the difference between the actual biological data signature (a) and predicted biological data signature (b).

In some aspects, the method can provide input vectors to a machine learning platform, wherein the machine learning platform outputs vectors that comprise components of a biological aging clock.

In some embodiments, a computer program product is provided on a tangible non-transitory computer readable medium that has a computer readable program code embodied therein, the program code being executable by a processor of a computer or computing system to perform a method as described herein.

In some embodiments, the methods can be performed for generating or determining a prognostic biomarker of aging for a patient. Such a method can include receiving a biological data signature derived from a patient tissue or organ (Step (a)). The method can include creating input vectors based on the biological data signature. The method can include providing input vectors to a machine learning platform (Step (b)). The method can include the machine learning platform generating output that includes a generated biological data signature given age of a sample from the patient tissue or organ (Step (c)). In some aspects, the prognostic biomarker of aging is specific to the tissue or organ, or specific to a characteristic of the tissue or organ. In some aspects, the machine learning platform includes the examples and embodiments thereof described herein or known in the art. The prognostic biomarker of aging can be considered a method that can be operated to generate a transcriptional signature given age of a tissue, organ, or subject, and then compare the predicted biological age with the actual age of the subject.

In some embodiments, the method performed by the computer program product can include repeating any Steps (a) (b) and (c) to create a second prognostic biomarker of aging. In some aspects, the two or more prognostic biomarkers of aging are combined to create a synthetic prognostic biomarker of aging that addresses the course of biological aging at the tissue, organ, or organism level. In some aspects, the method can include repeating Steps (a) and (b) a plurality of times to create a plurality prognostic biomarker of aging. In some aspects, the biological data signature of Step (a) and/or the profile of Step (b) is derived from a non-senescent tissue or organ of the patient or another organism.

The prognostic biomarker of aging can be developed using different methods/different tissues. In some instances, a prognostic biomarker of aging can be developed using biological data (e.g., transcriptomic data) extracted from blood profiles, or a biomarkers that was built for the skin tissues and blood. In the case of a ‘synthetic’ clock, there can be a generated biological data (transcriptional) signatures by multiple prognostic biomarker of aging that combined.

In some aspects, at least one of the biological data signatures (e.g., transcriptome signatures and/or proteome signature) is based on an in silico signaling pathway activation network decomposition, which is a decomposition performed with a machine learning platform, such as one described herein or otherwise known or created. The computational method can include any other computing steps described herein. The prognostic biomarker of aging can be specific to the tissue or organ, or specific to a characteristic of the tissue or organ.

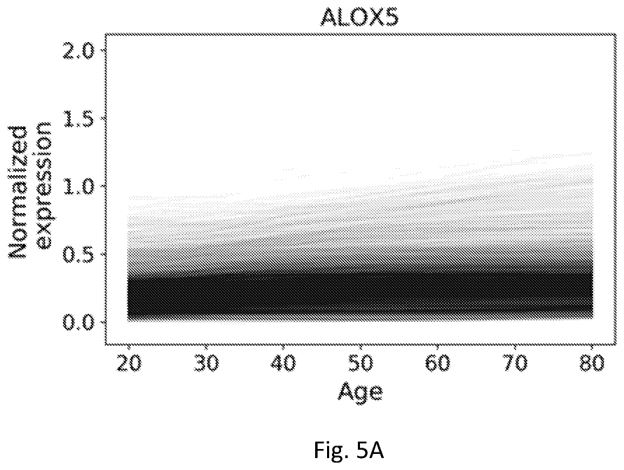

In some embodiments, the present technology relates to use of a generative neural network (GNN) that can be used to process biological data (e.g., biological data profile) of a subject and then to generate synthetic biological data for that subject for different biological ages of the subject. That is, the GNN produces predicted biological data profiles for the subject at a desired age point. For example, the subject may have a chronological age of 50, and the GNN processes this biological data signature in view of a target age, and then provides the synthetic biological data signature that is predicted for that subject at increased aging to a biological age of 60 (e.g., FIG. 4A) or at rejuvenation to a biological age of 45 (e.g., 4B) to show how the subject's biological profile would look at a younger age. Then, the information can be used to determine the health of the subject and identify protocols for reducing the aging process of that subject in an attempt to reduce help the subject obtain a younger biological age. The deep learning model provided herein can be used for prediction of the course of transcriptome ageing or rejuvenation.

In order to create and validate the model, gene expression profiles of whole blood were collected from a public domain (Gene Expression Omnibus). 10,000 blood transcriptome samples with chronological age (24 datasets) where collected in multiple countries (e.g., USA, UK, Estonia, Germany, Australia, Italy, Spain, Netherlands, and Singapore. The data was associated with the following meta information: Age, Sex, Race, and Batch ID. The GNN was configured based on the network proposed by Lample (Lample et al. “Fader Networks: Manipulating images by sliding attributes”, NIPS, 2017), and modified with an encoder (e.g., maps transcriptional profile to latent space representation) and a decoder (e.g., reconstruct transcriptome with given constrains). The iPANDA (Ozeror et al., “In Silico Pathway Activation Network Decomposition Analysis (iPANDA) as a method for biomarker development, Nature Communications, 2016) software suite was for signaling pathway analysis for 775 pathways from the NCI Pathway Interaction database.

In some embodiments, the GNN can be configured as a deep learning model that can be for analysis of biological data profiles of a subject and generation of synthetic biological data profiles for that subject, where the synthetic biological data profile is for a certain characteristic. For example, the synthetic biological profile can be the biological data profile of a certain biological age. While the synthetic biological profile can be based on transcriptional data profiles of the subject, other types of biological data may be used, such as those described herein. The GNN can produce: 1) generated biological data profiles (e.g., synthetic transcriptome samples) are personalized for a specific subject (e.g., subject providing the real biological samples); 2) heterogeneity in ageing changes of healthy individuals in the synthetic biological data profiles (e.g., transcriptomic level data profile) is significant and is preserved by the model; and 3) the proposed GNN model can be used to identify biological data (e.g., genes) and biological pathways associated with ageing.

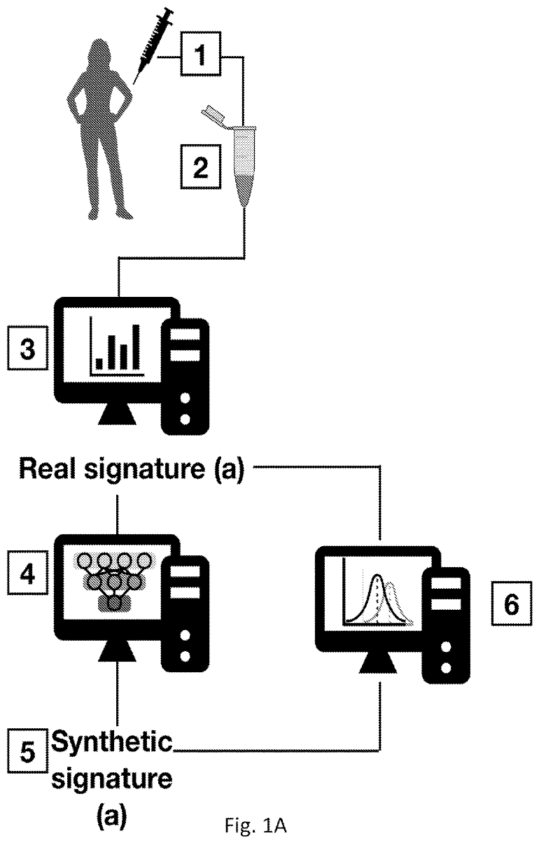

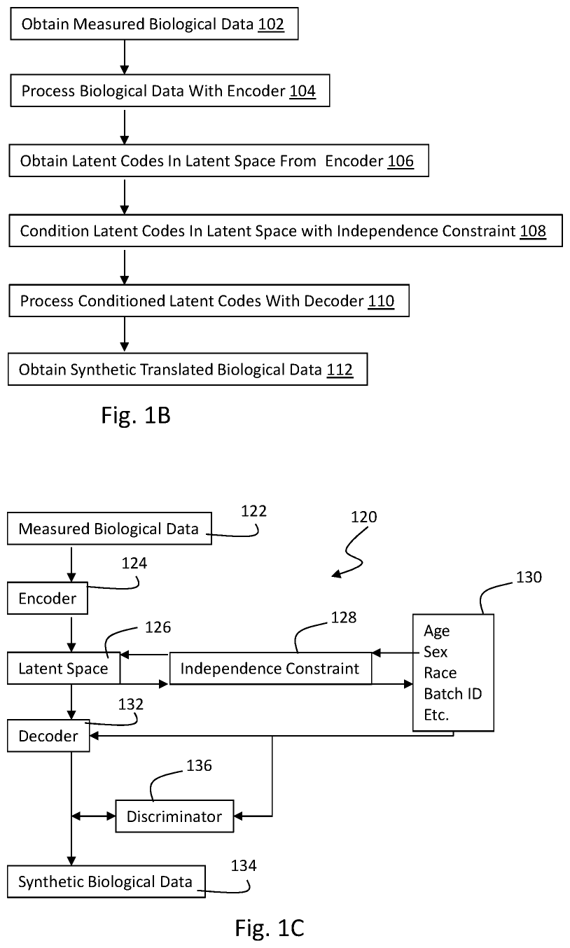

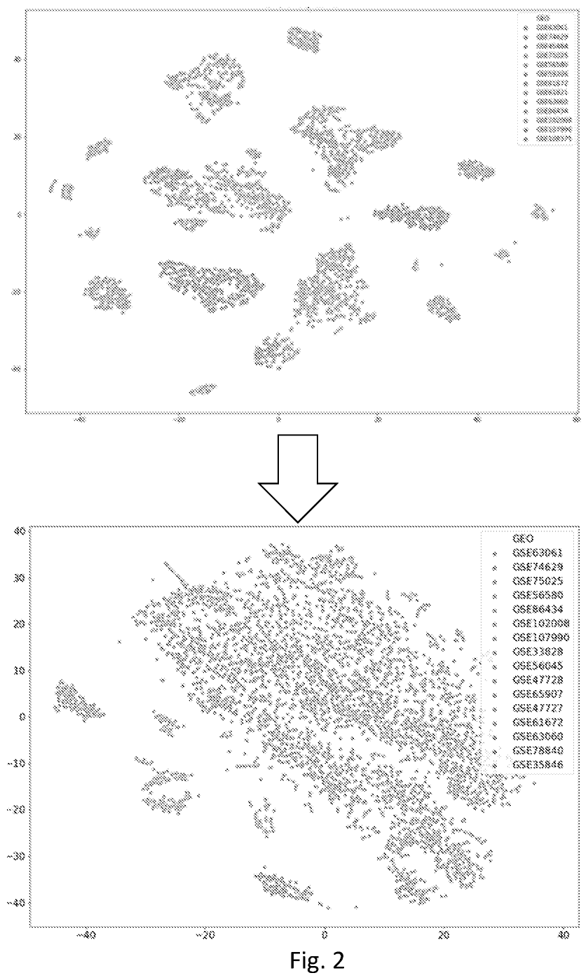

FIG. 1A illustrates an embodiment of a protocol in accordance with the present invention. The protocol includes obtaining a biological sample from a subject (1), and then processing the sample to obtain a biological data profile (2), such as by transcriptional profile of tissue, single cells or organ is profiled with measuring technique (RNA-Seq, microarray or Single-Cell RNA-Seq). A real signature for the biological data profile is calculated in a form of an absolute expression value of biological elements of the biological profile (e.g., genes and/or genetic elements), or in the form of a pathway signature (3). The real signature (e.g., transcriptional signature) is then used as an input vector to a generative model and processed (4). The generative model can serve as a prognostic aging biomarker analyzer. The synthetic signature of a synthetic biological data profile is generated by a GNN (5). Then, the synthetic signature is compared to the real signature to identify any differences therebetween (6). The identified differences are then identified as the biological target or biological pathway that contributes to the aging profile. These identified biological targets or biological pathways can then be analyzed for how to modulate them in order to reduce a biological age of the subject to reduce the synthetic biological age, and this information can be used for rejuvenation treatment.

FIG. 1B illustrates an embodiment of a method for the generation of the synthetic biological data profile. The method can include obtaining measured biological data from a subject at block 102. The measured biological can be processed as per item (3) of FIG. 1A, and then used as input into the GNN. The GNN then processes the measured biological data with an encoder at block 104, and latent codes in latent space are obtained from the encoder at block 106. The latent codes are conditioned with an independence constraint in the latent space at block 108. The independence constraint may be chronological age, sex, race batch ID, or other conditioning information or attribute of the subject (e.g., real or hypothetical attribute). The conditioned latent codes are then processed with a decoder at block 110, and synthetic translated biological data is then obtained at block 112. The synthetic translated biological data can provide the biological data profile for a defined biological age of the subject. For example, the proposed generative model can be used to produce synthetic transcriptome profiles given an age, sex, race and batch ID of the subject.

For example, the methods can include conditioning latent codes of the input vectors in a latent space of the machine learning platform with at least one constraint of an attribute of the subject, such that the predicted biological data signature is based on the at least one constraint.

In some embodiments, the encoder (e.g., a neural network) receives the real biological data that has a biological data signature, and then maps the biological data to a latent space representation. The decoder recreates a biological data signature from this latent space. The independence constraint functions as a discriminator over the latent space that can add conditions for recreation of a biological data signature for the same subject. For example, these conditions can be age, sex, ethnicity and etc. Therefore recreated synthetic biological data signature is generated with a specific condition.

FIG. 1C shows an embodiment of a GNN 120 that can perform the method of FIG. 1B. The GNN 120 can include an input of measured biological data 122 that is provided to an encoder 124 that produces the latent codes in the latent space 126. The latent codes in the latent space can be conditioned by an independence constraint 128, which can include information 130 about the subject that provided the measured biological data. The conditioned latent codes in the latent space can be processed by the decode 132 to provide the synthetic biological data 134. In some instances, a discriminator 136 functions as an independence constraint as over the latent space that can add conditions for recreation of a biological data signature for the same subject The GNN 120 may include the machine learning platform comprising one or more deep neural networks. The GNN 120 may include the machine learning platform comprising at least two generative adversarial networks and may comprise an adversarial autoencoder architecture. As for FIG. 1C, it is only one of the examples of how the generative model can be organized. The incorporated references provide other examples.

Any one of the method steps may be performed alone or in combination of other steps as recited herein. In some instances, the methods can include obtaining data and processing the data to obtain a recommendation for a treatment protocol. The recommended treatment protocol can then be implemented on the patient in accordance with parameters of the treatment protocol. That is, without the computational generation of the treatment protocol, the aspects of the treatment protocol cannot be performed without the instructions to do so. As such, obtaining the instructions, such as the type of drug and/or natural product or specific drug and/or natural product or combination of drugs and/or natural product, can be vital for performing the treatment protocol.

The biological data signature (e.g., transcriptome) may be based on a signature signaling pathway activation network analysis on a computer. One of the biological data signatures can be transcriptome signatures and/or proteome signatures that is based on in silico signaling pathway activation network decomposition. One of the profiles may comprise a Pearson correlation matrix.

In some embodiments, the personalized drug treatment determined from the protocols may comprise a senescence treatment for the patient. The profile of a biological data signature derived can be from a baseline, which may be derived from a non-senescent tissue or organ of the patient or another subject. The personalized drug treatment may be created by prescribing drugs identified by the classification vectors at their lowest effective dose.

The computer processing can include input and or processing of a complete or partial schematic overview of the biochemistry of senescence. Additional information can be obtained in the incorporated provisional application regarding the biological pathways that can be uses as input and processing for determining a treatment, such as specific drugs for the treatment. Accordingly, the biological pathways can be used in the methods described herein. Such biological pathways are described herein with some examples of computer processing thereof for implanting the design of treatment protocols as recited herein.

A variety of cell-intrinsic and-extrinsic stresses that can activate the cellular senescence program can be used as input for a simulation or other computer processing. The biological pathways that are known, such as in the literature, can be analyzed for specific biological steps that are performed. Modulation of the biological step either to increase the activity or decrease the activity results in a cascading series of events in response to the modulated activity. The modulations can be with drugs, substances, of other affirmative actions that effect a modulation of the biological pathway. This modulation can be measured for a defined biological step. The biological step and the change in response to the modulation activity can be used as inputs into computer models, and such computer models can be trained on the data. Now, with the increase in artificial intelligence and deep learning algorithms, such biological steps, the modulation activity, and the changed response can be used with such computer models for modeling biological pathways. This can allow for determining a modulation activity for one or more biological steps. Such modulations activities can be real and based on the simulations, such as being a real drug, substance, or medical action. The output of the computer models can be instructions or other information for causing the modulation activity in order to obtain a specific type of biological step modulation so that the end goal of a specifically modulated biological pathway can be obtained. Accordingly, the biological pathways described herein, or in the incorporated references and provisional applications, can be used as the biological pathways for the treatment protocols described herein.

To examine gene expression strategies that support the lifespan of different cell types within the human body, one can obtain available RNA-seq data sets and interrogated transcriptomes of various somatic cell types and tissues with reported cellular turnover, along with an estimate of lifespan, ranging from 2 days (monocytes) to effectively a lifetime (neurons). Across different cell lineages, one can obtain a gene expression signature of human cell and tissue turnover. In particular, turnover showed a negative correlation with the energetically costly cell cycle and factors supporting genome stability, concomitant risk factors for aging-associated pathologies.

Comparative transcriptome studies of long-lived and short-lived mammals, and analyses that examined the longevity trait across a large group of mammals (tissue-by-tissue surveys, focusing on brain, liver and kidney), have revealed candidate longevity-associated processes. Publicly available transcriptome data sets (for example, RNA-seq) generated by consortia, such as the Human Protein Atlas (HPA), or by The Genotype-Tissue Expression (GTEx) project or The Cancer Genome Atlas (TCGA) program can be used.

Methods for development of senescence drug treatments, that is, the selection of drugs, dosages, and cycles, are described herein. In this section, we give an overview of the drug treatments, themselves, that is, application of the personalized treatments once they have been designed, in a preferred embodiment, to the patient. In that patient, a tissue or organ is identified to which the senescent treatment will be applied.

In some embodiments, one phase of the treatment involves senoremediation, that is, a drug protocol of senoremediators, which are drugs that restore or increase the amount of presenescent cells (cells that are typical or a young, healthy tissue or organ). Another phase of the treatment involves senolytic treatment, that is, a drug protocol that involves restoring or that involves elimination or destruction of senescent cells in the tissue or organ of interest.

In some embodiments, one phase of the treatment involves an antifibrotic phase, that is, a drug protocol that addressing fibrotic cells in the tissue or organ of interest. Antifibrotic may involve restoring senescent cells to a pre-senescent, non-fibrotic state, elimination or destruction of fibrotic cells, or both.

A rating approach can be used to rank the senescence treating properties of treatments first involves collecting the transcriptome datasets from young and old patients and normalizing the data for each cell and tissue type, evaluating the pathway activation strength (PAS) for each individual pathway and constructing the pathway cloud and screen for drugs or combinations that minimize the signaling pathway cloud disturbance by acting on one or multiple elements of the pathway cloud. Drugs and combinations may be rated by their ability to return the signaling pathway activation pattern closer to that of the younger tissue samples. The predictions may be then tested both in vitro and in vivo on human cells and on model organisms such as rodents, nematodes and flies to validate the screening and rating algorithms. Pathway Activation and Pathway Activation Network Decomposition Analysis (iPANDA)(Ozerov et al., 2016), is a preferred method of network analysis for the methods described herein.

Development of senescence treatments (in particular drug combinations and protocols) as contemplated by the authors, are particularly compatible with the signaling pathway activation network analysis as described, for example, in US 2018/0125865 and Ozerov et. al., “In silico Pathway Activation Network Decomposition Analysis (iPANDA) as a method for biomarker development”, Nature Communications, 7: 13427, 2016, and both incorporated by specific reference in their entity. Such methods include large-scale transcriptomic data analysis that involves in silico Pathway Activation Network Decomposition Analysis (iPANDA). The capabilities of this method apply to multiple data sets containing data on obtained, for example, from Gene Expression Omnibus (GEO) or other biological data. Data sets in GEO are accessed by identifier, or accession number, such as GSE5350.

In a preferred embodiment, a deep neural network, similar to that described in, for example, Aliper et. al., “Deep learning applications for predicting pharmacological properties of drugs and drug repurposing using transcriptomic data”, Mol Pharm, 2016 Jul. 5; 13(7): 2524-2530, and Mamoshina et. al., “Applications of Deep Learning in Biomedicine”, Mol Pharm, 2016 Mar. 13(5), is used, in combination with a cellular signature database such as the LINCS database and a drug therapeutic use database such as MeSH, as inputs to the DNN in order to output drug classifications to develop a therapeutic protocol, in this case to categorize and choose drugs for a senescence or other treatment protocol. LINCS is the US Library of Network-Based Cellular Signatures Program aims to create a network-based understanding of biology by cataloging changes in gene expression and other cellular processes that occur when cells are exposed to a variety of perturbing agents. MeSH is (Medical Subject Headings) is the US National Library of Medicine controlled vocabulary thesaurus used for indexing articles for PubMed, the free search engine of references and abstracts on life sciences and biomedical topics also from the US National Library of Medicine.

An AAE works by matching the aggregated posterior to the prior ensures that generating from any part of prior space results in meaningful samples. As a result, the decoder of the adversarial autoencoder learns a deep generative model that maps the imposed prior to the data distribution. An AAE can be used in applications such as semi-supervised classification, disentangling style and content of images, unsupervised clustering, dimensionality reduction and data visualization. AAEs are used, for example, in generative modeling and semi-supervised classification tasks. Thus an AAE turns an autoencoder into a generative model. The AAE is often trained with dual objectives—a traditional reconstruction error criterion, and an adversarial training criterion that matches the aggregated posterior distribution of the latent representation of the autoencoder to an arbitrary prior distribution.

In a preferred embodiment derived from Kadurin, the method uses a 7-layer AAE architecture with the latent middle layer serving as a discriminator. As an input and output the AAE uses a vector of binary fingerprints and concentration of the molecule. In the latent layer we also introduced a neuron responsible for growth inhibition percentage, which when negative indicates the reduction in the number of tumor cells after the treatment. To train the AAE one uses a cell line assay data for compounds profiled in a cell line. The output of the AAE can then be used to screen drug compounds, such as the 72 million compounds in PubChem, and then select candidate molecules with potential anti-senescent or properties.

The latest class of non-parametric approaches for deep generative models is known as generative adversarial network (GAN). In this new framework, initially proposed by Goodfellow, generative models are estimated via an adversarial process. In practice, two models are simultaneously trained: a generative model G that captures the data distribution, and a discriminative model D that estimates the probability that a sample came from the training data rather than G. The training procedure for G is to maximize the probability of D making an error. Thus, this framework does not correspond to the standard optimization problem as it is based on a value function that one model seeks to maximize and the other seeks to minimize. The process terminates at a saddle point that is a minimum with respect to one model's strategy and a maximum with respect to the other model's strategy. Because GANs do not require an explicit representation of the likelihood, neither approximate inference nor Markov chains are necessary. Consequently, GANs provide an attractive alternative to maximum likelihood techniques.

Generative capabilities of deep adversarial network techniques open the doors to new perspectives as it could contribute to overcome several limitations of current data driven computational methods. For example, we can apply GANs on transcriptomics data for the generation of new samples for a desired phenotypic groups and in chemoinformatics for the prediction of the physical, chemical, or biological properties and structures of molecules. Quantitative structure—activity relationships (QSAR) and quantitative structure—property relationships (QSPR) are still considered as the modern standard for predicting properties of novel molecules. To that end, many ML-based approaches have been developed to tackle such problems, but recent results show that the DL-based methods match or outperform other state-of-the-art methods and demonstrate better predictive performance, parsimony and interpretability and web-based predictors are available on some cases. Furthermore, new methods based on convolutional neural networks are able to perform predictions by directly using graphs of arbitrary size and shape as inputs rather than fixed feature vectors and one can expect to see the development of more flexible deep generative architectures that can be applied directly to other structured data such as sequences, trees, graphs, and 3D structures. Thus, the deep adversarial network techniques could be used to improve accuracy, generative capabilities and predictive power and address several issues including computational cost, limited computation at each layer and limited information propagation across the graph.

Target prediction and mapping of bioactive small compounds and molecules by analyzing binding affinities and chemical properties is another area of research that makes extensive use of data-driven computational methods in order to optimize the use of data available in existing repositories. Despite promising results and the availability of web-platforms to computationally identify new targets for uncharacterized molecules or secondary targets for known molecules such as SwissTargetPrediction, in general, the available methods remain too inaccurate for systematic binding predictions and physical experiments remain the state of the art for binding determination. In this field, DL-based methods, such as the recently released methods AtomNet based on deep convolutional neural networks have allowed to circumvent several limitations and outperform more traditional computational methods including RFs, SVMs for QSAR and ligand-based virtual screening. One can expect that the development of DL-methods making use of the GAN framework will also lead to significant improvement with respect to prediction accuracy and power.

In some embodiments, the adversarial network and the autoencoder are trained jointly with SGD in two phases—the reconstruction phase and the regularization phase—executed on each mini-batch. In the reconstruction phase, the autoencoder updates the encoder and the decoder to minimize the reconstruction error of the inputs. In the regularization phase, the adversarial network first updates its discriminative network to tell apart the true samples (generated using the prior) from the generated samples (the hidden codes computed by the autoencoder). The adversarial network then updates its generator (which is also the encoder of the autoencoder) to confuse the discriminative network. Once the training procedure is done, the decoder of the autoencoder will define a generative model that maps the imposed prior of p(z) to the data distribution.

In some embodiments, the input layer is divided into a fingerprint part and a concentration input neuron. In some aspects, an AAE is trained to encode and reconstruct not only molecular fingerprints, but also experimental concentrations. The Encoder includes two consequent layers L1 and L2 with 128 and 64 neurons, respectively. The decoder includes the two layers L′1 and L′2, comprising 64 and 128 neurons respectively. The latent layer includes 5 neurons, one of which is the GI and the four others are discriminated with normal distribution. Since the protocol trains an encoder net to predict ‘efficiency’ against ‘senescence’ in a single neuron of latent layer, the latent vector is divided into two parts—‘GI’ and ‘representation’. A regression term is added to the encoder cost function. Furthermore, our encoder is restricted to map the same fingerprint to the same latent vector independently from input concentration by additional ‘manifold’ cost. The mean and variance of the concentrations is calculated through all dataset and then used to sample concentrations for ‘manifold’ step. On each step, the sample is fingerprinted from the training set and batch of concentration from normal distribution with given mean and variance. The training net with ‘manifold’ loss is performed by maximization of cosine similarity between ‘representations’ of similar fingerprints with different concentrations

All these changes resulted in a 5-step train iteration instead of a 3-step in AAE basic model: (a) Discriminator trained to distinguish between given latent distribution and encoded ‘representation’; (b) Encoder trained to confuse Discriminator with generated ‘representations’; (c) Encoder and Decoder trained jointly as Autoencoder; (d) Encoder trained to fit ‘score’ part of latent vector; (e) Encoder trained with ‘manifold’ cost.

The two first steps (a,b) are trained as usual adversarial networks. The Autoencoder cost function is computed as a sum of logloss of fingerprint part and mean squared error (MSE) of concentration parts and MSE is also used as a regression cost function. Example code for a preferred AAE is available at github.com/spoilt333/onco-aae.

### EXPERIMENTAL/SIMULATIONS/MODELS

Single Biopsy (or Existing Individual Profile).

Single biopsy test of liver or lung is taken from the patient according to standard procedures in medical center as described in in the nhlbi.hih.gov website. For a lung biopsy, few samples of lung tissue from several places in lungs will be taken. The samples are examined under a microscope, transcriptome and gene expression profiles and/or proteome and protein production profiles are also analyzed. This procedure can help rule out other conditions, such as sarcoidosis, cancer, or infection. Lung biopsy also can show how far disease has advanced.

There are several procedures to get lung tissue samples.

Video-assisted thoracoscopy. This is the most common procedure used to get lung tissue samples. An endoscope is inserted with an attached light and camera into chest through small cuts between ribs. The endoscope provides a video image of the lungs and allows to collect tissue samples. This procedure must be done in a hospital.

Bronchoscopy. For a bronchoscopy, a thin, flexible tube through is passed in nose or mouth, down a throat, and into airways. At the tube's tip are a light and mini-camera. They allow to see windpipe and airways. Then a forceps is inserted through the tube to collect tissue samples.

Bronchoalveolar lavage. During bronchoscopy, a small amount of salt water (saline) is injected through the tube into lungs. This fluid washes the lungs and helps bring up cells from the area around the air sacs. These cells are examined under a microscope.

Thoracotomy. For this procedure, a few small pieces of lung tissue are removed through a cut in the chest wall between ribs. Thoracotomy is done in a hospital.

For a liver biopsy, few samples of liver tissue from several places in liver will be taken. The samples are examined under a microscope, transcriptome and gene expression profiles are also analyzed.

There are several procedures to get live tissue samples.

Percutaneous Liver Biopsy. The health care provider either taps on the abdomen to locate the liver or uses one of the following imaging techniques: ultrasound or computerized tomography (CT) and will take samples with the needle.

Transvenous Liver Biopsy. When a person's blood clots slowly or the person has ascites—a buildup of fluid in the abdomen—the health care provider may perform a transvenous liver biopsy. A health care provider applies local anesthetic to one side of the neck and makes a small incision there, injects contrast medium into the sheath and take an x ray. After this insert and remove the biopsy needle several times if multiple samples are needed.

Laparoscopic Liver Biopsy. Health care providers use this type of biopsy to obtain a tissue sample from a specific area or from multiple areas of the liver, or when the risk of spreading cancer or infection exists. A health care provider may take a liver tissue sample during laparoscopic surgery performed for other reasons, including liver surgery.

Pathway Signature Measurement

Transcriptomic Data:

From the GEO database (ncbi.nlm.nih.gov/geo/) data sets containing gene expression data related to idiopathic pulmonary fibrosis (IPF) patients and normal healthy lung tissue used as a reference were downloaded (21 data sets). IPF and normal data from different data sets was preprocessed using GCRMA algorithm and summarized using updated chip definition files from Brainarray repository for each data set independently.

Differential genes were calculated using limma and deseq2 algorithms for groups of comparison: IPF (IPF vs reference healthy lung tissue); Senescence (old vs reference young healthy lung tissue); Smoking (current smoker vs reference non-smoker); Age status data was available for 2 data sets and smoking status data was available for 1 data set.

Differential expression genes data was used as an input for iPANDA algorithm in order to measure the pathway signature of each comparison group.

Pathway Database Overview:

There are several widely used collections of signaling pathways including Kyoto Encyclopedia of Genes and Genomes, QIAGEN and NCI Pathway Interaction Database.

In this study, we use the collection of signaling pathways most strongly associated with various types of malignant transformation in human cells obtained from the SABiosciences collection (sabiosciences.com/pathwaycentral.php).

Compare Signature Profiles.

Signature profile for each comparison group can be constructed based on iPANDA p-values cut-off (p-value <=0.05) and common overlap among different data sets: intersection cut-off threshold equal to 15 was used for IPF data, 2 for senescence data and 1 for smoking data.

Personalize the Treatment.

DNNs can be used as a tool to predict active compounds and generate a compounds with a desired efficacy. The application of DNN-based models can be used for personalization of compounds for individual patients and evaluation of the treatment efficacy and safety.

Machine learning approaches provide the tools of the analysis of biomedical data without prior assumption on the functional relations of this data. And Deep Neural Network (DNN) based approaches, such as multi-layered feed forward neural networks, are able to fit the complex and sparse biomedical data and learn highly non-linear dependencies of the raw data without the modification of features of interest. And deep learning is a state of the art method for many task from machine vision to language translation. But despite the fact, that biomedicine entered the era of “big data”, biomedical datasets are usually limited by sample sizes. And feature selection and dimensionality reduction of the feature space usually increase the predictive power of the DNNs applied in the biomedical domain (Aliper, Plis, et al. 2016).

A system can be provided that utilizes quantitative models with a deep architecture that is able to stratify compounds by their efficacy for the individual patient based his or her personal profile. In part, the personal profile can include the biological pathways analyzed with the quantitative models. The following data could be used as input feature to the system: gene expression profiles and signaling pathway profiles, blood tests (Putin et al. 2016), protein expression profiles, clinical history as well as a deep representation of the electronic health record (Miotto et al. 2016).

A system can be provided that utilizes the quantitative models with a deep architecture that is able to evaluate the efficacy of the proposed treatment through the quantitative assessment of the health status of the patient, such a biological age, life expectancy, the probability of survival. The following data could be used as input feature to the system: gene expression profiles and signaling pathway profiles, blood tests, protein expression profiles, clinical history as well as a deep representation of the electronic health record.

A system can be provided that utilizes the quantitative models with a deep architecture that is able to predict potential side effect of the treatment. The following data could be used as input feature to the system: gene expression profiles and signaling pathway profiles, blood tests, protein expression profiles, clinical history as well as a deep representation of the electronic health record.

A system can be provided based on generative model with deep architecture (Kadurin et al. 2017) that is able to generate molecules with a desired properties, such as high efficacy, low toxicity, high bioavailability and the like. Generated molecules can be evaluated by the DNN based systems through the efficacy and safety prediction.

### Examples

The invention includes methods, system, apparatus, computer program product, among others, to carry out the following.

No matter the particular type of biomarkers being assessed by a biological age assessment compatible with the current invention, a preferred embodiment of the deep learning computational approach for both the current invention and biological age assessment is as follows. A deep learning model is trained on blood expression profiles using the back-propagation algorithm. The proposed model is based on the assumption that the underlying dynamics of age-related gene expression changes depend on some individual for each sample latent features (z). The z is inferred from a single data point (x,y,s), where x is a vector of gene expression values, y is a chronological age, and s are other characteristics such as sex. The neural network G then defines the dynamics of the gene expression vector x=G(y;z,s). Denote the transition from age y to age y′ as:

T_{y→y′}^{s}(X)=D(E(x,y,s),y′,s).

A specific architecture of a deep learning model is based on the architecture published by Lample et al (Lample et al., 2017). The proposed deep learning model is a deep feed forward neural network trained with a loss function. An exemplary loss function expression is as follows:

$${min}_{E , G} {𝔼}_{( x , y ) ∼ {p}_{data}} [ {ℒ}_{identity} + {ℒ}_{perception} + {ℒ}_{independence} + {ℒ}_{consistency} + {ℒ}_{variation} ] .$$

Where:

1) Identity loss is a reconstruction loss stating that gene expression dynamics should pass through the point x at age y:

[symbol]

_{identity}(x,y,s)=∥x-T_{y→y}^{s}(x)∥².

2) Perception loss compares predicted and real age of a generated gene expression profile. We use an external pretrained age predictor P:

[symbol]

_{perception}(x,y,s)= [symbol] _{(x,y)˜Pdata}(P(T_{y→y}^{s}(x))−y)².

3) Independence loss encourages latent space z=E(x,y,s) to be independent of sex and other characteristics (s) and age (y) using adversarial learning: the protocol alternatively trains neural networks q_{y}(z) and qₛ(z) to predict y and s correspondingly and then train E to alter the predictor's performance (Lample et al., 2017). If no model can predict y and s better than a random predictor, z is independent of y and s.

$${ℒ}_{independence} ( x , y , s ) = {λ}_{{q}_{y}} {{min}_{{q}_{y}} ( {q}_{y} ( z ) - y )}^{2} + {λ}_{{q}_{s}} {min}_{{q}_{s}} {l}_{s} ( {q}_{s} ( z ) , s ) ,$$

where Iₛ is a loss function comparing predicted and real observed characteristics s.

4) The mapping z=E(x,y,s) is deterministic and the reconstruction loss encourages x=D(y;z, s). If at some point the dynamics do intersect at point x=D(y;z₁,s)=D(y;z₂,s), then E(x,y,s)=z₁=z₂. Hence, dynamics for different z should not intersect. However, since reconstruction, cycle consistency loss (Zhu et al., 2017) is added to prevent intersections. The model predicts gene expression x′ for a training object (x,y,$) at a random age y′. The model then infers z′ for a new object (x′,y′,s) and predict gene expression x″ at the original age y. If trajectories do not intersect, the error between the original and recovered objects should be close to zero:

[symbol]

_{consistency}= [symbol] _{(x,y)˜Pdata} [symbol] _{y′˜Pdata(y)}∥x−T_{y′→y}^{s}(T_{y→y′}^{s}(x))∥₂².

5) The final loss reduces variation in dynamics by penalizing non-monotonic behavior:

[symbol]

ᵥₐᵣᵢₐₜᵢₒₙ= [symbol] _{(x,y)˜Pdata}[|T_{y→y-1}^{s}(x)−T_{y→y}^{s}|+|T_{y→y+1}^{s}(x)−T_{y→y}^{s}(x)|−|T_{y→y+1}^{s}(x)−T_{y→y-1}^{s}(x)|].

Loss [symbol] ᵥₐᵣᵢₐₜᵢₒₙ is non-zero when dynamics are non-monotonic around y.

FIG. 1C illustrates the architecture of a preferred embodiment of the deep learning computational approach. The model comprises of an encoder 124 network and decoder 132 network. The encoder network receives a measured gene expression values in the form of a transcriptional signature and maps it to a latent space. The decoder network receives latent space vectors and target age along with other sample characteristics such as sex, race, and Batch ID and produces a translated or generated gene expression signature. Therefore recreated transcriptomic signature is generated with this condition.

The proposed deep learning model is a generative adversarial model, which alongside encoder and decoder networks has a discriminator 136 network, which is used to pass the age and other sample characteristics.

All of the networks in the model are trained simultaneously using a back propagation algorithm. The best architecture of the proposed model is selected by optimizing the loss function.

For example, this deep neural network is trained on 9560 gene expression profiles of whole blood liked to chronological age, health status, sex, and ethnicity and collected from a total of 20 datasets obtained from a public domain (Gene Expression Omnibus).

Table A provides a list of such datasets that were used to train a deep neural network in a preferred embodiment.

TABLE A

| Dataset identifier | Number of sample |
|---|---|
| GSE33828 | 881 |
| GSE56045 | 1202 |
| GSE47728 | 228 |
| GSE65907 | 2112 |
| GSE63061 | 388 |
| GSE74629 | 50 |
| GSE45484 | 120 |
| GSE75025 | 24 |
| GSE47727 | 122 |
| GSE56580 | 214 |
| GSE59206 | 198 |
| GSE61672 | 546 |
| GSE61821 | 402 |
| GSE63060 | 326 |
| GSE78840 | 653 |
| GSE86434 | 249 |
| GSE102008 | 733 |
| GSE107990 | 671 |
| GSE108375 | 313 |
| GSE35846 | 128 |

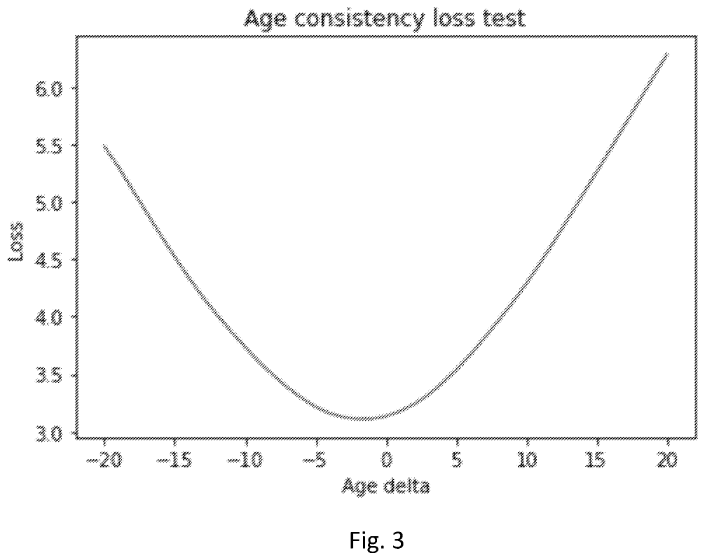

FIG. 2 illustrates the 2D representation of the personalized transcriptional vectors of the latent space for the generative model. Transformation of the real data (top) given the ID of the dataset eliminates the batch effect (bottom). Each dot on a plot represents individual samples. Samples generated given the dataset ID (bottom) show no clustering according to the Batch ID. The trained generative model eliminates batch effect in the datasets (FIG. 2), while preserving personalized features.

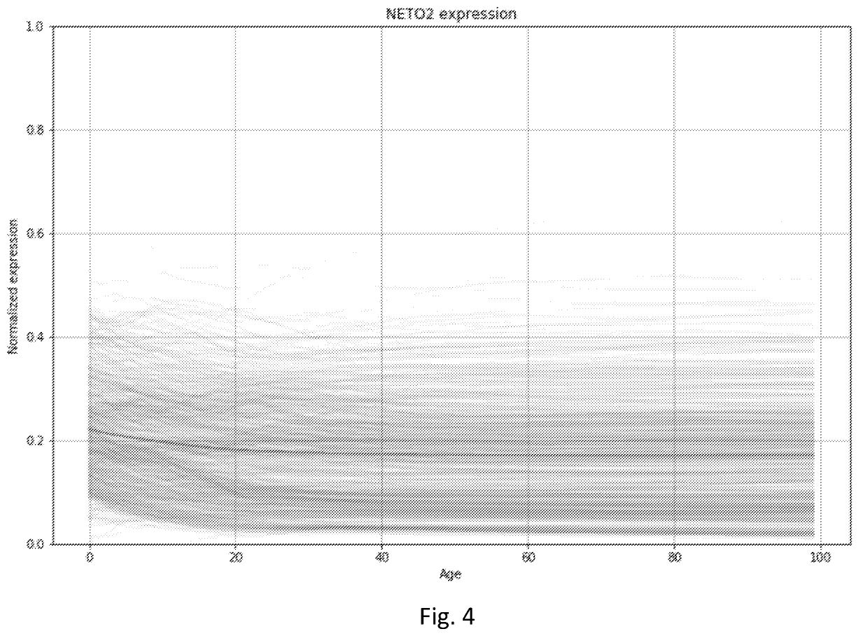

FIG. 3 illustrates the quality of the generated transcriptional profiles. As can be seen with a delta, such as difference between the actual and target age of 0 (target age is equal to the actual chronological age) the minimum deviation for the real age is observed (3.2 years). With an increased delta, the error rate is higher. The proposed model was also tasked to perform the generation of samples for multiple ages simultaneously. This way, continuous trajectories were obtained for individual genes. This way, generated age trajectories were analyzed for reference genes that are known to show constant expression levels in different tissue types, cells and routinely used in gene expression analysis (Caracausi et al., 2017).

FIG. 4 illustrates the age trajectories for a gene encoding Neuropilin And Tolloid Like 2 (NETO2) protein. As expected the age trajectory of the NETO2 is a constant function, which showing no age dependency. As such, the proposed model can be used to analyze age trajectories of previously uncharacterized genes.

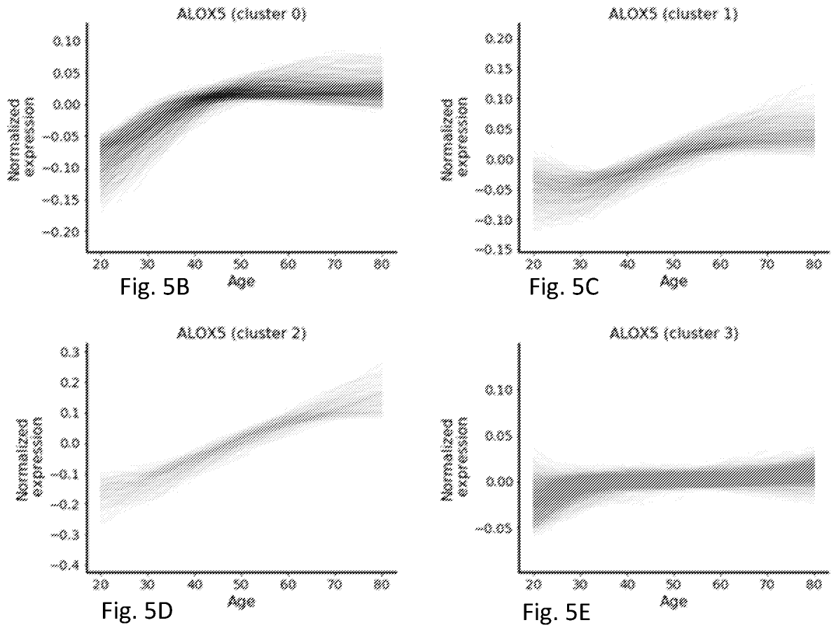

FIGS. 5A-5E illustrates the clustering of ALOX5 gene (e.g., encodes an enzyme that involved in the biosynthesis of proteins that regulates the inflammation process in the human body) age trajectories shows four categories of expression profiles. Cluster 0 trajectories show an increase in expression levels before 50 years and constant expression levels after 50 years. Cluster 1 trajectories show a monotonic increase of expression levels. Cluster 2 trajectories show a faster increase of expression levels compared to the cluster 1. Cluster 3 trajectories show constant expression levels. Suggesting that age-related changes are not universal among people. And that there is a lot of heterogeneity.

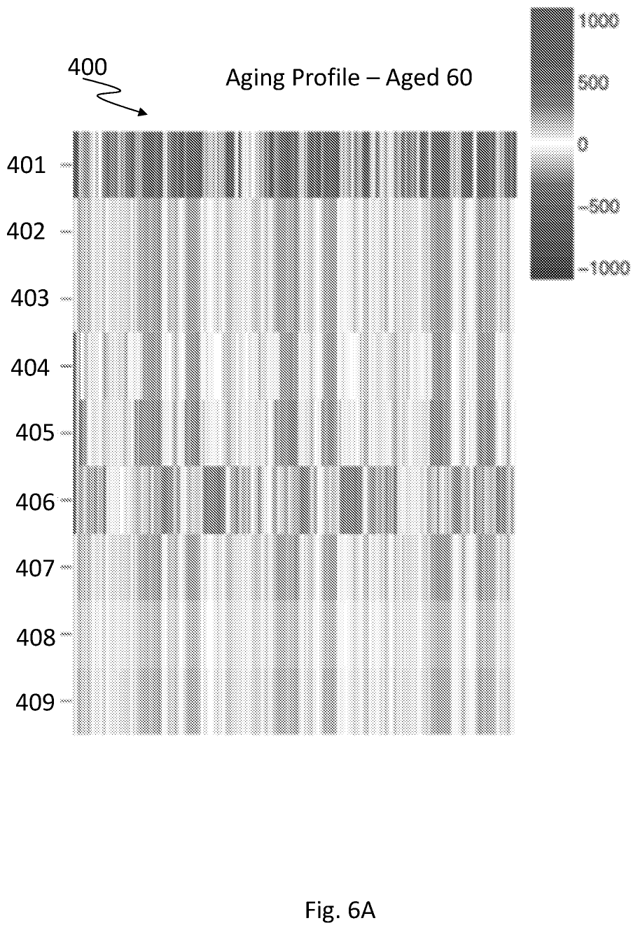

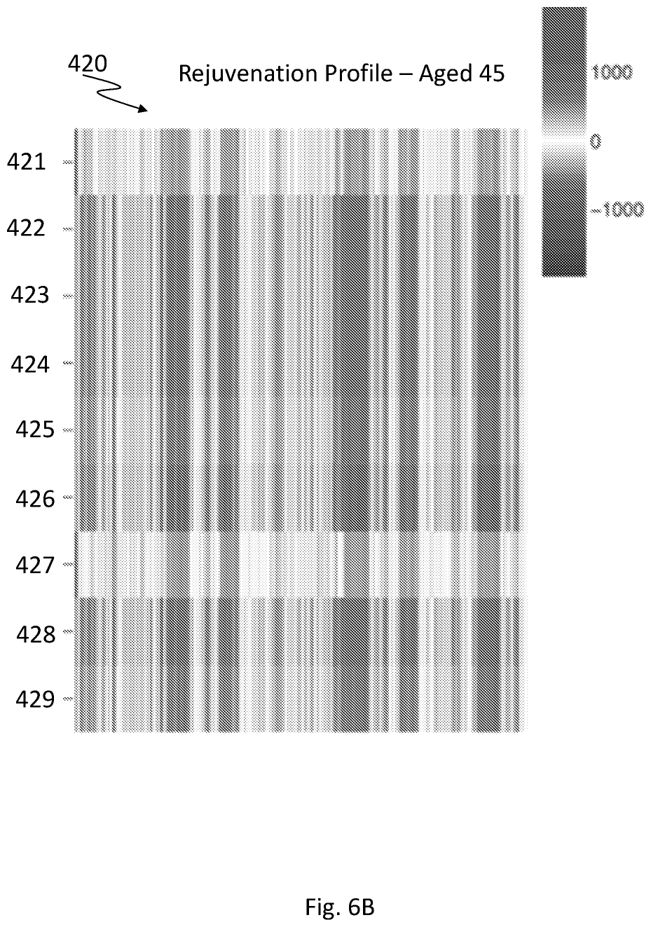

Further analysis of the generated transcriptome profiles showed that signaling pathways vary among individuals (FIGS. 5A-5E). For example, ageing trajectories Arachidonate 5-Lipoxygenase (ALOX5) gene vary significantly between individuals. The same was observed on signaling pathway level. At the same time, there are common pathways between ‘rejuvenation’ (−15 years) and ‘ageing’ (+15 years) including ‘Signaling events mediated by VEGFR1 and VEGFR2’ and ‘Thromboxane A2 receptor signaling’, as shown in FIGS. 6A-6B.

FIGS. 6A-6B illustrate the top 9 signaling pathways perturbed by the ‘rejuvenation’ (FIG. 6B) and ‘aging’ (FIG. 6A). Up and down-regulated genes (pathways) are shown in red and green respectively, which are in greyscale. The saturation of the color denotes to the perturbation amplitude. The data indicates that there are common pathways between ‘rejuvenation’ (−15 years) and ‘ageing’ (+15 years) including ‘Signaling events mediated by VEGFR1 and VEGFR2’ and ‘Thromboxane A2 receptor signaling’. iPANDA (Ozerov et al., 2016) software was used for signaling pathway analysis and 775 pathways from the NCI Pathway Interaction database.

Thus, the present invention provides a deep learned model for a generation of transcriptional data. The results show that 1) generated transcriptome samples are personalized 2) heterogeneity in ageing changes of healthy individuals on the transcriptomic level is significant and is preserved by the model, and 3) the proposed model can be used to identify genes and pathways associated with ageing.

The invention can provide a model in aging research and/or treatment. At the same time, it also can be used to remove some sensitive information from signatures. For example, if one wants for some reason to remove the ethnicity from the transcriptomic signature, it can be easily done with such a model. As such, any characteristic, such as those recited herein, can be removed from the model.

The figures provided herein are examples of reports or can be included in reports of the synthetic biological data. The reports can be provided to the subject or a medical professional, such as the subject's doctor.

In some embodiments, the biological data signature is based on genomics, transcriptomics, proteomics, methylomics, metabolomics, lipidomics, glycomics, or secretomics. In some aspects, the method includes obtaining biological sample of the tissue or organ of the subject; and obtaining the biological data by performing a measurement of the genomics, transcriptomics, proteomics, metabolomics, lipidomics, glycomics, or secretomics. In some aspects, the biological data signature is based on a simulation by a computer program for genomics, transcriptomics, proteomics, methylomics, metabolomics, lipidomics, glycomics, or secretomics. In some aspects, the biological data is an omics signature of biological data. In some aspects, the omics signature is genomics, transcriptomics, proteomics, metabolomics, methylomics, lipidomics, glycomics, or secretomics.

The use of genomics, transcriptomics, and proteomics (e.g., biological data signatures) in the present protocols for determining biological aging clocks and other protocols are described above. These protocols can also be applied to other biomarkers or other omics, where the omics may be considered to also be biomarkers.

Genomics is the study of the structure, function, evolution, mapping, and editing of genomes. A genome is an organism's complete set of DNA, including all of its genes. In contrast to genetics, which refers to the study of individual genes and their roles in inheritance, genomics aims at the collective characterization and quantification of all of an organism's genes, their interrelations and influence on the organism. As such, genomics provides the biological data signature for use in preparing the biological aging clocks and other protocols described herein. The genes may direct the production of proteins with the assistance of enzymes and messenger molecules. In turn, proteins make up body structures such as organs and tissues as well as control chemical reactions and carry signals between cells. Accordingly, the genomics biological data signature can provide significant information. Genomics also involves the sequencing and analysis of genomes through uses of high throughput DNA sequencing and bioinformatics to assemble and analyze the function and structure of entire genomes.

Transcriptomics is the study of the transcriptome, which is the set of all RNA transcripts, including coding and non-coding, in an individual or a population of cells. The term can also sometimes be used to refer to all RNAs, or just mRNA, depending on the particular experiment. The term transcriptome is a portmanteau of the words transcript and genome; it is associated with the process of transcript production during the biological process of transcription. The study of the transcriptome can provide biological data signatures for the cells, tissues, or organs or the overall organism. This data can be used as described herein.

Proteomics is the study of proteins in the proteome, which can obtain a biological data signature of the proteins in cells, fluids, tissues, organs, or a subject. The proteome is the entire set of proteins that is produced or modified by an organism or system. Proteomics has enabled the identification of ever increasing numbers of proteins, and protein levels. The protein signature varies with time and distinct requirements, or stresses, that a cell or organism undergoes.

The metabolomics includes the study of chemical processes involving metabolites, the small molecule substrates, intermediates and products of metabolism. Specifically, metabolomics is the systematic study of the unique chemical fingerprints that specific cellular processes leave behind, the study of their small-molecule metabolite profiles. As such, metabolomics can be studied to obtain a signature from a tissue or organ of a subject. The metabolome represents the complete set of metabolites in a biological cell, tissue, organ or organism, which are the end products of cellular processes. The mRNA gene expression data and proteomic analyses reveal the set of gene products being produced in the cell, data that represents one aspect of cellular function. Conversely, metabolic profiling and obtaining biological data signatures thereof can give an instantaneous snapshot of the physiology of that cell, and thus, metabolomics provides a direct functional readout of the physiological state of an organism. This biological data signature of metabolomics can provide for the information for creating the biological aging clocks and other protocols as described herein. Also, the protocols can be used to integrate genomics, transcriptomic, proteomic, and metabolomic information to provide a better understanding of cellular biology and creation of the biological aging clock and other protocols.

The lipidomics is the study of pathways and networks of cellular lipids in biological systems, which can provide a biological data signature of the lipids. The word lipidome is used to describe the complete lipid profile within a cell, tissue, organism, or ecosystem and is a subset of the metabolome, which also includes the three other major classes of biological molecules: proteins/amino-acids, sugars and nucleic acids. Lipidomics is can be assessed by techniques such as mass spectrometry (MS), nuclear magnetic resonance (NMR) spectroscopy, fluorescence spectroscopy, dual polarization interferometry and computational methods. Also, the biological data signature of the lipidomics can be used for determination of a biological aging clock due to the role of lipids in many metabolic diseases such as obesity, atherosclerosis, stroke, hypertension and diabetes.

The glycomics is the study of glycomes, which includes the entire complement of sugars, whether free or present in more complex molecules of an organism, including genetic, physiologic, pathologic, and other aspects. Glycomics is the systematic study of all glycan structures of a given cell type or organism and is a subset of glycobiology. Accordingly, glycomics gives biological data signatures of the glycan structures, which can be used in the protocols and biological aging clocks described herein. The term glycomics is derived from the chemical prefix for sweetness or a sugar, “glyco-”, and was formed to follow the omics naming convention established by genomics (which deals with genes) and proteomics (which deals with proteins).

Secretomics is a study that involves the analysis of the secretome, which includes all the secreted proteins of a cell, tissue or organism. Secreted proteins are involved in a variety of physiological processes, including cell signaling and matrix remodeling, but are also integral to invasion and metastasis of malignant cells. Secretomics has been especially important in the discovery of biomarkers for cancer and understanding molecular basis of pathogenesis. Accordingly, secretomics can be used to obtain a biological data signature for the cells, fluids, tissues, organs, and organisms, which can be useful for determining biological aging clocks and other protocols described herein.

Methylomics is a study that involves the analysis of methylome, which includes nucleic acid modification of the organism's genome. Methylation leads to epigenetic modifications of DNA and so reduction of gene expression and consequently protein synthesis. Such epigenetic modifications are involved in the regulation of many biological processes inside cells including aging. Decreased methylation is associated with aging of tissue and cells. Methylation data gives biological data signatures, which can be used in biological aging clocks and other protocols described herein.

For the processes and methods disclosed herein, the operations performed in the processes and methods may be implemented in differing order. Furthermore, the outlined operations are only provided as examples, and some operations may be optional, combined into fewer operations, eliminated, supplemented with further operations, or expanded into additional operations, without detracting from the essence of the disclosed embodiments.

The figures provided herein are examples of reports or can be included in reports of the biological synthetic sample and synthetic characteristics. The reports can be provided to the subject or a medical professional, such as the subject's doctor.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only, and is not intended to be limiting.

In one embodiment, the present methods can include aspects performed on a computing system. As such, the computing system can include a memory device that has the computer-executable instructions for performing the methods. The computer-executable instructions can be part of a computer program product that includes one or more algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

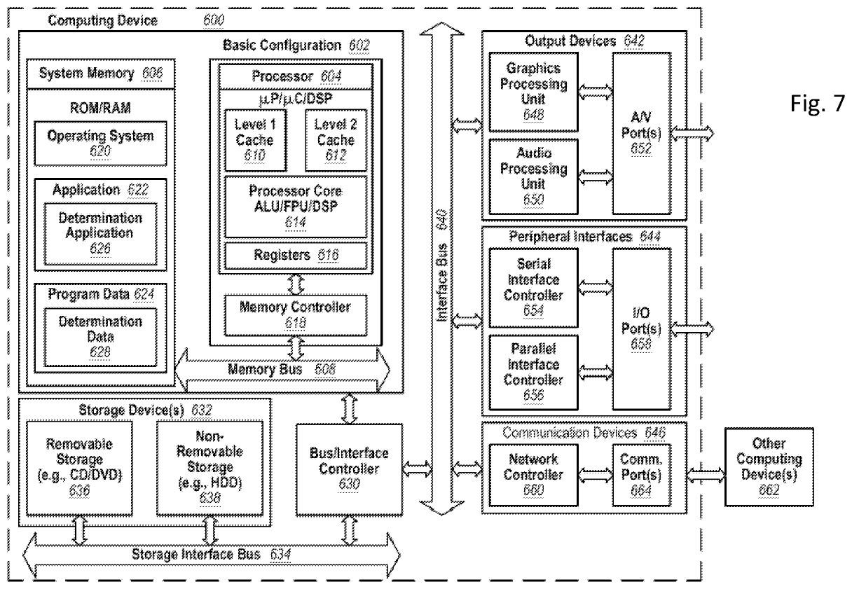

FIG. 7 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

This patent cross-references: U.S. application Ser. No. 16/415,855 filed May 17, 2019, U.S. application Ser. No. 16/104,391 filed Aug. 17, 2018, U.S. application Ser. No. 16/044,784 filed Jul. 25, 2018, U.S. Provisional Application No. 62/536,658 filed Jul. 25, 2017, and U.S. Provisional Application No. 62/547,061 filed Aug. 17, 2017, which applications are incorporated herein by specific reference in their entirety.

All references recited herein are incorporated herein by specific reference in their entirety.

### REFERENCES

Buzdin, et. al., US 2017/0073735
Goodfellow et. al., “Generative Adversarial Networks”, arXiv:1406.2661v1, 2014.
Makhzani et. al., “Adversarial Autoencoders”, arXiv:1511.05644v2, 2015.
Kadurin, et. al., “The cornucopia of meaningful leads: Applying deep adversarial autoencoders for new molecule development in oncology”, Oncotarget, 2017, Vol. 8, (No. 7), pp: 10883-10890.
Seim et. al., “Gene expression signatures of human cell and tissue longevity”, npj Aging and Mechanisms of Disease, 2, 16014 (2016).
Ozerov, U.S. 62/401,789, filed September 2016.
Aliper et. al., “Deep learning applications for predicting pharmacological properties of drugs and drug repurposing using transcriptomic data”, Mol Pharm, 2016 Jul. 5; 13(7): 2524-2530.
Mamoshina et. al., “Applications of Deep Learning in Biomedicine”, Mol Pharm, 2016 Mar. 13(5),
Ozerov et. al., “In silico Pathway Activation Network Decomposition Analysis (iPANDA) as a method for biomarker development”, Nature Communications, 7:13427, 2016.
Munoz-Espin, D., & Serrano, M. (2014). Cellular senescence: from physiology to pathology. Nature reviews Molecular cell biology, 15(7), 482-496.
Acosta, Juan Carlos, Ana Banito, Torsten Wuestefeld, Athena Georgilis, Peggy Janich, Jennifer P. Morton, Dimitris Athineos, et al. 2013. “A Complex Secretory Program Orchestrated by the Inflammasome Controls Paracrine Senescence.” Nature Cell Biology 15 (8): 978-90.
Baar, Marjolein P., Renata M. C. Brandt, Diana A. Putavet, Julian D. D. Klein, Kasper W. J. Derks, Benjamin R. M. Bourgeois, Sarah Stryeck, et al. 2017. “Targeted Apoptosis of Senescent Cells Restores Tissue Homeostasis in Response to Chemotoxicity and Aging.” Cell 169 (1): 132-47.e16.
Baker, Darren J., Robbyn L. Weaver, and Jan M. van Deursen. 2013. “p21 Both Attenuates and Drives Senescence and Aging in BubR1 Progeroid Mice.” Cell Reports 3 (4): 1164-74.
Caracausi, M., Piovesan, A., Antonaros, F., Strippoli, P., Vitale, L., and Pelleri, M. C. (2017). Systematic identification of human housekeeping genes possibly useful as references in gene expression studies. Mol. Med. Rep. 16, 2397-2410.
Campisi, Judith. 2005. “Senescent Cells, Tumor Suppression, and Organismal Aging: Good Citizens, Bad Neighbors.” Cell 120 (4): 513-22.
Campisi J. Cellular senescence: putting the paradoxes in perspective. Current opinion in genetics & development. 2011; 21(1):107-112. doi. 10.1016/j.gde 2010.10.005.
Campisi J. Aging, Cellular Senescence, and Cancer. Annual review of physiology. 2013; 75:685-705. doi:10.1146/annurev-physiol-030212-183653. Campisi, Judith, and Fabrizio d'Adda di Fagagna. 2007. “Cellular Senescence: When Bad Things Happen to Good Cells.” Nature Reviews. Molecular Cell Biology 8 (9): 729-40.
Chilosi, Marco, Angelo Carloni, Andrea Rossi, and Venerino Poletti. 2013. “Premature Lung Aging and Cellular Senescence in the Pathogenesis of Idiopathic Pulmonary Fibrosis and COPD/emphysema.” Translational Research: The Journal of Laboratory and Clinical Medicine 162 (3): 156-73.
Chilosi, Marco, Alberto Zamo, Claudio Doglioni, Daniela Reghellin, Maurizio Lestani, Licia Montagna, Serena Pedron, et al. 2006. “Migratory Marker Expression in Fibroblast Foci of Idiopathic Pulmonary Fibrosis.” Respiratory Research 7 (1). doi: 10.1186/1465-9921-7-95.
Coppé, Jean-Philippe, Christopher K. Patil, Francis Rodier, Yu Sun, Denise P. Munoz, Joshua Goldstein, Peter S. Nelson, Pierre-Yves Desprez, and Judith Campisi. 2008. “Senescence-Associated Secretory Phenotypes Reveal Cell-Nonautonomous Functions of Oncogenic RAS and the p53 Tumor Suppressor.” PLoS Biology 6 (12): 2853-68.
De Cecco M, Criscione S W, Peckham E J, et al. Genomes of replicatively senescent cells undergo global epigenetic changes leading to gene silencing and activation of transposable elements Aging cell. 2013; 12(2):247-256. doi:10.1111/acel.12047.
Demaria M, Ohtani N, Youssef S A, et al. An Essential Role for Senescent Cells in Optimal Wound Healing through Secretion of PDGF-AA. Developmental cell. 2014; 31(6):722-733. doi:10.1016/j.devce1.2014.11.012.
Deursen, Jan M. van. 2014. “The Role of Senescent Cells in Ageing.” Nature 509 (7501): 439-46.
DiLoreto, R., and C. T. Murphy. 2015. “The Cell Biology of Aging.” Molecular Biology of the Cell 26 (25): 4524-31.
Freund, Adam, Arturo V. Orjalo, Pierre-Yves Desprez, and Judith Campisi. 2010. “Inflammatory Networks during Cellular Senescence: Causes and Consequences.” Trends in Molecular Medicine 16 (5): 238-46.
Galkin, F., Mamoshina, P., Aliper, A., Putin, E., Moskalev, V., Gladyshev, V. N., and Zhavoronkov, A. (2020). Human gut microbiome aging clock based on taxonomic profiling and deep learning. IScience 23, 101199.
Vestbo, J. et al. Global strategy for the diagnosis, management, and prevention of chronic obstructive pulmonary disease: GOLD executive summary. Am. J. Respir. Crit. Care Med. 187, 347-365 (2013).
Hannum, G., Guinney, J., Zhao, L., Zhang, L., Hughes, G., Sadda, S., Klotzle, B., Bibikova, M., Fan, J.-B., Gao, Y., et al. (2013). Genome-wide Methylation Profiles Reveal Quantitative Views of Human Aging Rates. Mol. Cell 49, 359-367.
Hernandez Gea, Virginia, and Scott L. Friedman. 2011. “Pathogenesis of Liver Fibrosis. Annual Review of Pathology: Mechanisms of Disease 6 (1): 425-56.
Ivanov, Andre, Jeff Pawlikowski, Indrani Manoharan, John van Tuyn, David M. Nelson, Taranjit Singh Rai, Parisha P. Shah, et al. 2013. “Lysosome-Mediated Processing of Chromatin in Senescence.” The Journal of Cell Biology 202 (1): 129-43.
Jun, Joon-Il, and Lester F. Lau. 2010. “The Matricellular Protein CCN1 Induces Fibroblast Senescence and Restricts Fibrosis in Cutaneous Wound Healing.” Nature Cell Biology 12 (7): 676-85.
Kim, William Y., and Norman E. Sharpless. 2006. “The Regulation of INK4/ARF in Cancer and Aging.” Cell 127 (2): 265-75.
Krimpenfort, Paul, and Anton Berns. 2017. “Rejuvenation by Therapeutic Elimination of Senescent Cells.” Cell 169 (1): 3-5.
Krishnamurthy, Janakiraman, Matthew R. Ramsey, Keith L. Ligon, Chad Torrice, Angela Koh, Susan Bonner-Weir, and Norman E. Sharpless. 2006. “p16INK4a Induces an Age-Dependent Decline in Islet Regenerative Potential.” Nature 443 (7110): 453-57.
Krizhanovsky, Valery, Monica Yon, Ross A. Dickins, Stephen Hearn, Janelle Simon, Cornelius Miething, Herman Yee, Lars Zender, and Scott W. Lowe. 2008. “Senescence of Activated Stellate Cells Limits Liver Fibrosis.” Cell 134 (4): 657-67.
Kuwano, K., R. Kunitake, M. Kawasaki, Y. Nomoto, N. Hagimoto, Y. Nakanishi, and N. Hara. 1996. “P21Waf1/Cip1/Sdi1 and p53 Expression in Association with DNA Strand Breaks in Idiopathic Pulmonary Fibrosis.” American Journal of Respiratory and Critical Care Medicine 154 (2 Pt 1): 477-83.
Laberge, Remi-Martin, Pierre Awad, Judith Campisi, and Pierre-Yves Desprez. 2012. “Epithelial-Mesenchymal Transition Induced by Senescent Fibroblasts.” Cancer
Microenvironment: Official Journal of the International Cancer Microenvironment Society 5 (1): 39-44.
Lomas, Nicola J., Keira L. Watts, Khondoker M. Akram, Nicholas R. Forsyth, and Monica A. Spiteri. 2012. “Idiopathic Pulmonary Fibrosis: Immunohistochemical Analysis Provides Fresh Insights into Lung Tissue Remodelling with Implications for Novel Prognostic Markers.” International Journal of Clinical and Experimental Pathology 5 (1): 58-71.
Malavolta, Marco, Elisa Pierpaoli, Robertina Giacconi, Laura Costarelli, Francesco Piacenza, Andrea Basso, Maurizio Cardelli, and Mauro Provinciali. 2016. “Pleiotropic Effects of Tocotrienols and Quercetin on Cellular Senescence: Introducing the Perspective of Senolytic Effects of Phytochemicals.” Current Drug Targets 17 (4): 447-59.
Mallette, Frederick A., and Gerardo Ferbeyre. 2007. “The DNA Damage Signaling Pathway Connects Oncogenic Stress to Cellular Senescence.” Cell Cycle 6 (15): 1831-36.
Minagawa, S., J. Araya, T. Numata, S. Nojiri, H. Hara, Y. Yumino, M. Kawaishi, et al. 2010. “Accelerated Epithelial Cell Senescence in IPF and the Inhibitory Role of SIRT6 in TGF-Induced Senescence of Human Bronchial Epithelial Cells.” AJP: Lung Cellular and Molecular Physiology 300 (3): L391-401.
Muñoz-Espin, Daniel, Marta Cañamero, Antonio Maraver, Gonzalo Gómez-López, Julio Contreras, Silvia Murillo-Cuesta, Alfonso Rodriguez-Baeza, et al. 2013. “Programmed Cell Senescence during Mammalian Embryonic Development.” Cell 155 (5): 1104-18.
Polina Mamoshina, Kirill Kochetov, Evgeny Putin, Franco Cortese, Alexander Aliper, Won-Suk Lee, Sung-Min Ahn, Lee Uhn, Neil Skjodt, Olga Kovalchuk, Morten Scheibye-Knudsen, Alex Zhavoronkov; Population Specific Biomarkers of Human Aging: A Big Data Study Using South Korean, Canadian, and Eastern European Patient Populations, The Journals of Gerontology: Series A, gly005, doi.org/10.1093/gerona/gly005.
Mamoshina, P., Volosnikova, M., Ozerov, I. V, Putin, E., Skibina, E., Cortese, F., and Zhavoronkov, A. (2018b). Machine Learning on Human Muscle Transcriptomic Data for Biomarker Discovery and Tissue-Specific Drug Target Identification. Front. Genet. 9, 242.
Mamoshina, P., Kochetov, K., Cortese, F., Kovalchuk, A., Aliper, A., Putin, E., Scheibye-Knudsen, M., Cantor, C. R., Skjodt, N. M., Kovalchuk, O., et al. (2019). Blood Biochemistry Analysis to Detect Smoking Status and Quantify Accelerated Aging in Smokers. Sci. Rep. 9, 142.
Nelson, Glyn, James Wordsworth, Chunfang Wang, Diana Jurk, Conor Lawless, Carmen Martin-Ruiz, and Thomas von Zglinicki. 2012. “A Senescent Cell Bystander Effect: Senescence-Induced Senescence.” Aging Cell 11 (2): 345-49.
Nikolich-Zugich, Janko. 2008. “Ageing and Life-Long Maintenance of T-Cell Subsets in the Face of Latent Persistent Infections.” Nature Reviews. Immunology 8 (7): 512-22.
Noble, Paul W., Carlo Albera, Williamson Z. Bradford, Ulrich Costabel, Marilyn K. Glassberg, David Kardatzke, Talmadge E. King Jr, et al. 2011. “Pirfenidone in Patients with Idiopathic Pulmonary Fibrosis (CAPACITY): Two Randomised Trials.” The Lancet 377 (9779): 1760-69.
Ohtani, Naoko, Kimi Yamakoshi, Akiko Takahashi, and Eiji Hara. 2004. “The p16INK4a-RB Pathway: Molecular Link between Cellular Senescence and Tumor Suppression.” The Journal of Medical Investigation: JMI 51 (3,4): 146-53.
Ozerov, Ivan V., Ksenia V. Lezhnina, Evgeny Izumchenko, Artem V. Artemov, Sergey Medintsev, Quentin Vanhaelen, Alexander Aliper, et al. 2016. “In Silico Pathway Activation Network Decomposition Analysis (iPANDA) as a Method for Biomarker Development.” Nature Communications 7 (November): 13427.
Parrinello, Simona, Jean-Philippe Coppe, Ana Krtolica, and Judith Campisi. 2005. “Stromal-Epithelial Interactions in Aging and Cancer: Senescent Fibroblasts Alter Epithelial Cell Differentiation.” Journal of Cell Science 118 (Pt 3): 485-96.
Putin, E., Mamoshina, P., Aliper, A., Korzinkin, M., and Moskalev, A. (2016). Deep biomarkers of human aging: Application of deep neural networks to biomarker development. 8, 1-13.
Seki, Ekihiro, and David A. Brenner. 2015. “Recent Advancement of Molecular Mechanisms of Liver Fibrosis.” Journal of Hepato-Biliary-Pancreatic Sciences 22 (7): 512-18.
Seki, Ekihiro, and Robert F. Schwabe. 2015. “Hepatic Inflammation and Fibrosis: Functional Links and Key Pathways.” Hepatology 61 (3): 1066-79.
Storer, Mekayla, Alba Mas, Alexandre Robert-Moreno, Matteo Pecoraro, M. Carmen Ortells, Valeria Di Giacomo, Reut Yosef, et al. 2013. “Senescence Is a Developmental Mechanism That Contributes to Embryonic Growth and Patterning.” Cell 155 (5): 1119-30.
Takeuchi, Shinji, Akiko Takahashi, Noriko Motoi, Shin Yoshimoto, Tomoko Tajima, Kimi Yamakoshi, Atsushi Hirao, et al. 2010. “Intrinsic Cooperation between p16INK4a and p21Waf1/Cip1 in the Onset of Cellular Senescence and Tumor Suppression in Vivo.” Cancer Research 70 (22): 9381-90.
Wang, Jianrong, Glenn J. Geesman, Sirkka Liisa Hostikka, Michelle Atallah, Benjamin Blackwell, Elbert Lee, Peter J. Cook, et al. 2011. “Inhibition of Activated Pericentromeric SINE/Alu Repeat Transcription in Senescent Human Adult Stem Cells Reinstates Self-Renewal.” Cell Cycle 10 (17): 3016-30.
Li, Yifeng, Chih-Yu Chen, and Wyeth W. Wasserman. “Deep feature selection: Theory and application to identify enhancers and promoters.” International Conference on Research in Computational Molecular Biology. Springer International Publishing, 2015.
Yacoub, Meziane, and Y. Bennani. “HVS: A heuristic for variable selection in multilayer artificial neural network classifier.” Intelligent Engineering Systems Through Artificial Neural Networks, St. Louis, Mo. Vol. 7. 1997.
Dorizzi, B., et al. “Variable selection using generalized RBF networks: Application to the forecast of the French T-bonds.” CESA′96 IMACS Multiconference: computational engineering in systems applications. 1996.
Refenes, A. P. N., A. D. Zapranis, and J. Utans. “Neural model identification variable selection and model adequacy.” Decision Technologies for Financial Engineering, Proceedings of NNCM 96. 1998.
Ruck, Dennis W., Steven K. Rogers, and Matthew Kabrisky. “Feature selection using a multilayer perceptron.” Journal of Neural Network Computing 2.2 (1990): 40-48.
Czernichow, Thomas. “Architecture selection through statistical sensitivity analysis.” International Conference on Artificial Neural Networks. Springer Berlin Heidelberg, 1996.
Lehmann, G., Muradian, K. K., & Fraifeld, V. E. (2013). Telomere length and body temperature—independent determinants of mammalian longevity?. Frontiers in genetics, 4.
Wolters, S., & Schumacher, B. (2013). Genome maintenance and transcription integrity in aging and disease. Frontiers in genetics, 4.
Horvath, S., Zhang, Y., Langfelder, P., Kahn, R. S., Boks, M. P., van Eijk, K., & Ophoff, R. A. (2012). Aging effects on DNA methylation modules in human brain and blood tissue. Genome Biol, 13(10), R97.
Horvath, S. (2013). DNA methylation age of human tissues and cell types. Genome biology, 14(10), R115.
Mendelsohn, A. R., & Larrick, J. W. (2013). The DNA Methylome as a biomarker for epigenetic instability and human aging. Rejuvenation research, 16(1), 74-77.
Chowers, I., Liu, D., Farkas, R. H., Gunatilaka, T. L., Hackam, A. S., Bernstein, S. L., . . . & Zack, D. J. (2003). Gene expression variation in the adult human retina. Human molecular genetics, 12(22), 2881-2893.
Weindruch, R., Kayo, T., Lee, C. K., & Prolla, T. A. (2002). Gene expression profiling of aging using DNA microarrays. Mechanisms of ageing and development, 123(2), 177-193.
Park, S. K., Kim, K., Page, G. P., Allison, D. B., Weindruch, R., & Prolla, T. A. (2009). Gene expression profiling of aging in multiple mouse strains: identification of aging biomarkers and impact of dietary antioxidants. Aging cell, 8(4), 484-495.
Zahn, J. M., Poosala, S., Owen, A. B., Ingram, D. K., Lustig, A., Carter, A., & Becker, K. G. (2007). AGEMAP: a gene expression database for aging in mice. PLoS genetics, 3(11), e201.
Blalock, E. M., Chen, K. C., Sharrow, K., Herman, J. P., Porter, N. M., Foster, T. C., & Landfield, P. W. (2003). Gene microarrays in hippocampal aging: statistical profiling identifies novel processes correlated with cognitive impairment. The Journal of neuroscience, 23(9), 3807-3819.
Welle, S., Brooks, A. I., Delehanty, J. M., Needler, N., & Thornton, C. A. (2003). Gene expression profile of aging in human muscle. Physiological genomics, 14(2), 149-159.
Park, S. K., & Prolla, T. A. (2005). Gene expression profiling studies of aging in cardiac and skeletal muscles. Cardiovascular research, 66(2), 205-212.
Hong, M. G., Myers, A. J., Magnusson, P. K., & Prince, J. A. (2008). Transcriptome-wide assessment of human brain and lymphocyte senescence. PLoS One, 3(8), e3024.
de Magalhães, J. P., Curado, J., & Church, G. M. (2009). Meta-analysis of age-related gene expression profiles identifies common signatures of aging. Bioinformatics, 25(7), 875-881.
Zhavoronkov, A., & Cantor, C. R. (2011). Methods for structuring scientific knowledge from many areas related to aging research. PloS one, 6(7), e22597.
Trindade, L. S., Aigaki, T., Peixoto, A. A., Balduino, A., da Cruz, I. B. M., & Heddle, J. G. (2013). A novel classification system for evolutionary aging theories. Frontiers in genetics, 4.
Putin, E. et al. (2016) Deep biomarkers of human aging: Application of deep neural networks to biomarker development. Aging 8(5):1021-1033.
Lavecchia, A. and Cerchia, C. (2016) In silico methods to address polypharmacology: current status, applications and future perspectives. Drug Discov. Today 21(2):288-298.
Oquab, M. et al. (2014) Learning and Transferring Mid-level Image Representations Using Convolutional Neural Networks. 2014 IEEE Conference on Computer Vision and Pattern Recognition [Internet]. IEEE. 1717-24. doi:10.1109/CVPR.2014.222.
Ma, J. et al. (2015) Deep Neural Nets as a Method for Quantitative Structure-Activity Relationships. J Chem Inf Model. 55(2):263-74.
Wang, C. et al. (2014) Pairwise Input Neural Network for Target-Ligand Interaction Prediction.Bioinformatics and Biomedicine (BIBM), 2014 IEEE International Conference. 67-70.
Xu, Y. et al. (2015) Deep Learning for Drug-Induced Liver Injury. J. Chem. Inf. Model. 55 (10):2085-2093. doi:10.1021/acs.jcim.5b00238
Hughes, T. B. et al. (2015) Modeling Epoxidation of Drug-like Molecules with a Deep Machine Learning Network. ACS Cent Sci. 1(4):168-80. doi:abs/10.1021/acscentsci.5b00131
Mayr, A. et al. (2016) DeepTox: Toxicity Prediction using Deep Learning. Frontiers in Environmental Science. doi:10.3389/fenvs.2015.00080
Aliper, Alexander, Aleksey V. Belikov, Andrew Garazha, Leslie Jellen, Artem Artemov, Maria Suntsova, Alena Ivanova, et al. 2016. “In Search for Geroprotectors: In Silico Screening and in Vitro Validation of Signalome-Level Mimetics of Young Healthy State.” Aging 8 (9): 2127-52.
Aliper, Alexander M., Antonei Benjamin Csoka, Anton Buzdin, Tomasz Jetka, Sergey Roumiantsev, Alexey Moskalev, and Alex Zhavoronkov. 2015. “Signaling Pathway Activation Drift during Aging: Hutchinson-Gilford Progeria Syndrome Fibroblasts Are Comparable to Normal Middle-Age and Old-Age Cells.” Aging 7 (1). Impact Journals, LLC: 26.
Ansari, Habib R., Ahmed Nadeem, M. A. Hassan Talukder, Shilpa Sakhalkar, and S. Jamal Mustafa. 2007. “Evidence for the Involvement of Nitric Oxide in A2B Receptor-Mediated Vasorelaxation of Mouse Aorta.” American Journal of Physiology. Heart and Circulatory Physiology 292 (1): H719-25.
Astarita, Giuseppe, Kwang-Mook Jung, Vitaly Vasilevko, Nicholas V. Dipatrizio, Sarah K. Martin, David H. Cribbs, Elizabeth Head, Carl W. Cotman, and Daniele Piomelli. 2011. “Elevated Stearoyl-CoA Desaturase in Brains of Patients with Alzheimer's Disease.” PloS One 6 (10): e24777.
Bobrov, E., Georgievskaya, A., Kiselev, K., Sevastopolsky, A., Zhavoronkov, A., Gurov, S., Rudakov, K., Del Pilar Bonilla Tobar, M., Jaspers, S., and Clemann, S. (2018). PhotoAgeClock: deep learning algorithms for development of non-invasive visual biomarkers of aging. Aging (Albany. N.Y.). 10, 3249-3259.
Campbell L, Saville C R, Murray P J, Cruickshank S M, Hardman M J. Local Arginase 1 Activity Is Required for Cutaneous Wound Healing. The Journal of Investigative Dermatology. 2013; 133(10):2461-2470. doi:10.1038/jid.2013.164.
Cole J J, Robertson N A, Rather M I, et al. Diverse interventions that extend mouse lifespan suppress shared age-associated epigenetic changes at critical gene regulatory regions. Genome Biology. 2017; 18:58. doi:10.1186/s13059-017-1185-3.
Colegio, Oscar R., Ngoc-Quynh Chu, Alison L. Szabo, Thach Chu, Anne Marie Rhebergen, Vikram Jairam, Nika Cyrus, et al. 2014. “Functional Polarization of Tumour-Associated Macrophages by Tumour-Derived Lactic Acid.” Nature 513 (7519): 559-63.
Deignan, Joshua L., Justin C. Livesay, Paul K. Yoo, Stephen I. Goodman, William E. O'Brien, Ramaswamy K. Iyer, Stephen D. Cederbaum, and Wayne W. Grody. 2006. “Ornithine Deficiency in the Arginase Double Knockout Mouse.” Molecular Genetics and Metabolism 89 (1-2): 87-96.
Douarre, Celine, Carole Sourbier, Ilaria Dalla Rosa, Benu Brata Das, Christophe E. Redon, Hongliang Zhang, Len Neckers, and Yves Pommier. 2012. “Mitochondrial Topoisomerase I Is Critical for Mitochondrial Integrity and Cellular Energy Metabolism.” PloS One 7 (7). Public Library of Science. doi:10.1371/journal.pone.0041094.
Gosule, L. C., and J. A. Schellman. 1976. “Compact Form of DNA Induced by Spermidine.” Nature 259 (5541): 333-35.
Khiati, Salim, Simone A. Baechler, Valentina M. Factor, Hongliang Zhang, Shar-Yin N. Huang, Ilaria Dalla Rosa, Carole Sourbier, Leonard Neckers, Snorri S. Thorgeirsson, and Yves Pommier. 2015. “Lack of Mitochondrial Topoisomerase I (TOP1mt) Impairs Liver Regeneration.” Proceedings of the National Academy of Sciences of the United States of America 112 (36): 11282-87.
Kunduri, S. S., S. J. Mustafa, D. S. Ponnoth, G. M. Dick, and M. A. Nayeem. 2013. “Adenosine A1 Receptors Link to Smooth Muscle Contraction via CYP4a, PKC-α, and ERK1/2.” Journal of Cardiovascular Pharmacology 62 (1). NIH Public Access: 78.
Madauss, Kevin P., William A. Burkhart, Thomas G. Consler, David J. Cowan, William K. Gottschalk, Aaron B. Miller, Steven A. Short, Thuy B. Tran, and Shawn P. Williams. 2009. “The Human ACC2 CT-Domain C-Terminus Is Required for Full Functionality and Has a Novel Twist.” Acta Crystallographica. Section D, Biological Crystallography 65 (5): 449-61.
Maesaka, John K., Bali Sodam, Thomas Palaia, Louis Ragolia, Vecihi Batuman, Nobuyuki Miyawaki, Shubha Shastry, Steven Youmans, and Marwan El-Sabban. 2013. “Prostaglandin D2 Synthase: Apoptotic Factor in Alzheimer Plasma, Inducer of Reactive Oxygen Species, Inflammatory Cytokines and Dialysis Dementia.” Journal of Nephropathology 2 (3): 166-80.
Magalhães, Joao Pedro de, Joao Curado, and George M. Church. 2009. “Meta-Analysis of Age-Related Gene Expression Profiles Identifies Common Signatures of Aging.” Bioinformatics 25 (7): 875-81.
Mak, Isabella Wy, Nathan Evaniew, and Michelle Ghert. 2014. “Lost in Translation: Animal Models and Clinical Trials in Cancer Treatment.” American Journal of Translational Research 6 (2): 114-18.
Ma, Yina, and Ji Li. 2015. “Metabolic Shifts during Aging and Pathology.” Comprehensive Physiology 5 (2): 667-86.
McKinnon, Peter J. 2016. “Topoisomerases and the Regulation of Neural Function.” Nature Reviews. Neuroscience 17 (11): 673-79.
Moskalev A, Et al. 2017. “Geroprotectors.org: A New, Structured and Curated Database of Current Therapeutic Interventions in Aging and Age-Related Disease. —PubMed—NCBI.” Accessed March 17. ncbi.nlm.nih.gov/pubmed/26342919.
Nozaki, Hiroaki, Taisuke Kato, Megumi Nihonmatsu, Yohei Saito, Ikuko Mizuta, Tomoko Noda, Ryoko Koike, et al. 2016. “Distinct Molecular Mechanisms of HTRA1 Mutants in Manifesting Heterozygotes with CARASIL.” Neurology 86 (21): 1964-74.
Ogneva, Irina V., Nikolay S. Biryukov, Toomas A. Leinsoo, and Irina M. Larina. 2014. “Possible Role of Non-Muscle Alpha-Actinins in Muscle Cell Mechanosensitivity.” PloS One 9 (4). Public Library of Science: e96395.
Peters, M. J., Joehanes, R., Pilling, L. C., Schurmann, C., Conneely, K. N., Powell, J., Reinmaa, E., Sutphin, G. L., Zhernakova, A., Schramm, K., et al. (2015). The transcriptional landscape of age in human peripheral blood. Nat. Commun. 6, 8570.
Petkovich D A, Podolskiy D I, Lobanov A V, Lee S-G, Miller R A, Gladyshev V N. Using DNA methylation profiling to evaluate biological age and longevity interventions. Cell metabolism. 2017; 25(4):954-960.e6. doi:10.1016/j.cmet.2017.03.016.
Phillips, Catherine M., Louisa Goumidi, Sandrine Bertrais, Martyn R. Field, L. Adrienne Cupples, Jose M. Ordovas, Jolene McMonagle, et al. 2010. “ACC2 Gene Polymorphisms, Metabolic Syndrome, and Gene-Nutrient Interactions with Dietary Fat.” Journal of Lipid Research 51 (12): 3500-3507.
Pinto, Elisabete. 2007. “Blood Pressure and Ageing.” Postgraduate Medical Journal 83 (976). BMJ Group: 109.
Pledgie, Allison, Yi Huang, Amy Hacker, Zhe Zhang, Patrick M. Woster, Nancy E. Davidson, and Robert A. Casero Jr. 2005. “Spermine Oxidase SMO(PAOh1), Not N1-Acetylpolyamine Oxidase PAO, Is the Primary Source of Cytotoxic H₂O₂ in Polyamine Analogue-Treated Human Breast Cancer Cell Lines.” The Journal of Biological Chemistry 280 (48): 39843-51.
Qian, Hao, Na Luo, and Yuling Chi. 2012. “Aging-Shifted Prostaglandin Profile in Endothelium as a Factor in Cardiovascular Disorders.” Journal of Aging Research 2012 (February). Hindawi Publishing Corporation. doi:10.1155/2012/121390.
Savolainen, Kalle, Tiina J. Kotti, Werner Schmitz, Teuvo I. Savolainen, Raija T. Sormunen, Mika Ilves, Seppo J. Vainio, Ernst Conzelmann, and J. Kalervo Hiltunen. 2004. “A Mouse Model for Alpha-Methylacyl-CoA Racemase Deficiency: Adjustment of Bile Acid Synthesis and Intolerance to Dietary Methyl-Branched Lipids.” Human Molecular Genetics 13 (9): 955-65.
Selkälä Eija M., Remya R. Nair, Werner Schmitz, Ari-Pekka Kvist, Myriam Baes, J. Kalervo Hiltunen, and Kaij a J. Autio. 2015. “Phytol Is Lethal for Amacr-Deficient Mice.” Biochimica et Biophysica Acta 1851 (10): 1394-1405.
Sergio Solórzano-Vargas, R., Diana Pacheco-Alvarez, and Alfonso Leon-Del-Rio. 2002. “Polycarboxylate Synthetase Is an Obligate Participant in Biotin-Mediated Regulation of Its Own Expression and of Biotin-Dependent Carboxylases mRNA Levels in Human Cells.” Proceedings of the National Academy of Sciences of the United States of America 99 (8). National Academy of Sciences: 5325-30.
Suzuki, Yoichi, Xue Yang, Yoko Aoki, Shigeo Kure, and Yoichi Matsubara. 2005. “Mutations in the monocarboxylate Synthetase Gene HLCS.” Human Mutation 26 (4): 285-90.
Tang, Eva H. C., and Paul M. Vanhoutte. 2008. “Gene Expression Changes of Prostanoid Synthases in Endothelial Cells and Prostanoid Receptors in Vascular Smooth Muscle Cells Caused by Aging and Hypertension.” Physiological Genomics 32 (3): 409-18.
Thomas, Inas, and Brigid Gregg. 2017. “Metformin; a Review of Its History and Future: From Lilac to Longevity.” Pediatric Diabetes 18 (1): 10-16.
Thomas, T., and T. J. Thomas. 2017. “Polyamine Metabolism and Cancer. —PubMed—NCBI.” Accessed April 11. ncbi.nlm.nih.gov/pubmed/12927050.
Tong, Liang. 2013. “Structure and Function of Biotin-Dependent Carboxylases.” Cellular and Molecular Life Sciences: CMLS 70 (5). NIH Public Access: 863.
Unno, Keiko, Tomokazu Konishi, Aimi Nakagawa, Yoshie Narita, Fumiyo Takabayashi, Hitomi Okamura, Ayane Hara, et al. 2015. “Cognitive Dysfunction and Amyloid β Accumulation Are Ameliorated by the Ingestion of Green Soybean Extract in Aged Mice.” Journal of Functional Foods 14: 345-53.
Verdura E, Et al. 2017. “Heterozygous HTRA1 Mutations Are Associated with Autosomal Dominant Cerebral Small Vessel Disease.—PubMed—NCBI.” Accessed April 11. ncbi.nlm.nih.gov/pubmed/26063658.
Weller J, Et al. 2017. “Age-Related Decrease of Adenosine-Mediated Relaxation in Rat Detrusor Is a Result of A2B Receptor Downregulation.—PubMed—NCBI.” Accessed April 17. ncbi.nlm.nih.gov/pubmed/25728851.
Zhang, Yongyou, Amar Desai, Sung Yeun Yang, Ki Beom Bae, Monika I. Antczak, Stephen P. Fink, Shruti Tiwari, et al. 2015. “TISSUE REGENERATION. Inhibition of the Prostaglandin-Degrading Enzyme 15-PGDH Potentiates Tissue Regeneration.” Science 348 (6240): aaa2340.
Seim, Inge, Siming Ma, and Vadim N. Gladyshev. 2016. “Gene Expression Signatures of Human Cell and Tissue Longevity.” Npj Aging and Mechanisms of Disease 2 (1). doi:10.1038/npjamd.2016.14.
Lample et al. ‘Fader Networks: Manipulating images by sliding attributes’. NIPS 2017.
Ozerov et al. ‘In silico Pathway Activation Network Decomposition Analysis (iPANDA) as a method for biomarker development’. Nature Communications 2016.
Zhu, J.-Y., Park, T., Isola, P., and Efros, A. A. (2017). Unpaired Image-to-Image Translation using Cycle-Consistent Adversarial Networks. In Computer Vision (ICCV), 2017 IEEE International Conference On, p.

1. A method of creating synthetic biological data for a subject, the method comprising:
(a) receiving a real biological data signature derived from a biological sample of the subject;
(b) creating input vectors based on the real biological data signature;
(c) inputting the input vectors into a machine learning platform;
(d) generating a predicted biological data signature of the subject based on the input vectors by the machine learning platform, wherein the predicted biological data signature includes synthetic biological data specific to the subject; and
(e) preparing a report that includes the synthetic biological data of the subject.

2. The method of claim 1, further comprising:
creating at least a second biological data signature by repeating any one or more of steps (a), (b), (c), and/or (d), wherein the second biological data signature is based on a second real biological data signature from the biological sample of the subject, a different biological sample of the subject, or a second biological sample of a second subject; and
optionally, preparing a report that includes a second synthetic biological data of the second biological data signature.

3. The method of claim 1, further comprising:
comparing the predicted biological data signature with the real biological data signature of the subject;
determining a difference between the synthetic biological data of the subject with the real biological sample of the subject; and
preparing the report with that identifies the difference between the synthetic biological data with the real biological sample of the subject.

4. The method of claim 3, further comprising identification of at least one biomarker having a difference between the synthetic biological data with the real biological sample of the subject.

5. The method of claim 4, further comprising identifying at least one biological target, wherein modulation of the at least one biological target modulates the identified at least one biomarker.

6. The method of claim 1, wherein the real biological data signature is based on biological pathway activation signatures for genomics, transcriptomics, proteomics, metabolomics, lipidomics, glycomics, methylomics, or secretomics, and the predicted biological data corresponds with the biological activation signature.

7. The method of claim 6, further comprising at least one of:
correlating a genomics profile with the predicted biological data signature of the subject;
correlating a proteomics profile with the predicted biological data signature of the subject;
correlating a transcriptomics profile with the predicted biological data signature of the subject;
correlating a metabolomics profile with the predicted biological data signature of the subject;
correlating a lipidomics profile with the predicted biological data signature of the subject;
correlating a glycomics profile with the predicted biological data signature of the subject;
correlating a secretomics profile with the predicted biological data signature of the subject; or
correlating a methylomics profile with the predicted biological data signature of the subject.

8. The method of claim 1, further comprising:
performing feature importance analysis for ranking biological data by importance in age prediction by using the real biological data signature; and
identifying a subset biomarkers of the biological pathway activation signature thereof that are selected as indicators of a condition of the subject.

9. The method of claim 8, further comprising identifying at least one biological target associated with the condition, wherein modulation of the at least one biological target modulates at least one biomarker of the identified subset of biological markers.

10. The method of claim 1, further comprising correlating the predicted biological data signature with a predicted biological age of the subject.

11. The method of claim 1, further comprising:
obtaining the biological sample from the subject; and
obtaining the real biological data signature by performing a measurement of the genomics, transcriptomics, proteomics, metabolomics, lipidomics, glycomics, methylomics, or secretomics.

12. The method of claim 11, wherein the predicted biological data signature is based on a simulation by a computer program for biological pathway activation signatures for genomics, transcriptomics, proteomics, metabolomics, lipidomics, glycomics, methylomics, or secretomics.

13. The method of claim 1, further comprising conditioning latent codes of the input vectors in a latent space of the machine learning platform with at least one constraint of an attribute of the subject, such that the predicted biological data signature is based on the at least one constraint.

14. The method of claim 13, wherein the synthetic biological data is for a defined biological age of the subject, wherein the predicted biological data signature represents a biological data signature of the subject at the defined biological age.

15. The method of claim 1, wherein the synthetic biological data is for one of:
an aging simulation to increase a biological age of the biological data signature of the subject; or
a rejuvenation simulation to decrease a biological age of the biological data signature of the subject.

16. The method of claim 15, further comprising identification of at least one biomarker having a difference between the real biological sample of the subject with the biological data signature of the aging simulation or the rejuvenation simulation.

17. The method of claim 16, further comprising identifying at least one biological target, wherein modulation of the at least one biological target modulates the identified at least one biomarker.

18. The method of claim 1, after a defined time period,
performing steps (a), (b), (c), (d), and (e) in a second iteration;
comparing the initial report with the report of the second iteration; and
determining a change in the predicted biological data signature over the defined time period.

19. The method of claim 18, further comprising identification of at least one biomarker having a difference over the defined time period.

20. The method of claim 19, further comprising identifying at least one biological target, wherein modulation of the at least one biological target modulates the identified at least one biomarker.

21. The method of claim 18, further comprising:
determining an aging rate over the defined time period based on the change in the predicted biological data signature; and
tracking the change in the predicted biological data signature over the defined time period.

22. The method of claim 1, further comprising:
performing a therapeutic regimen over a defined time period,
performing steps (a), (b), (c), (d), and (e) in a second iteration;
comparing the initial report with the report of the second iteration;
determining a change in the predicted biological data signature over the defined time period; and
determining:
whether the therapeutic regimen changed the predicted biological data signature,
if the therapeutic regimen changed the predicted biological data signature, then determine whether or not to: continue therapeutic regimen, change therapeutic regimen, or stop therapeutic regimen, or
if the therapeutic regimen does not change the predicted biological data signature, then determine whether or not to: continue therapeutic regimen, change therapeutic regimen, or stop therapeutic regimen.

23. The method of claim 1 wherein the predicted biological data signature is generated based on at least one attribute of the subject, wherein the attribute is selected from age, sex, tissue types, ethnicity, life expectancy, or combination thereof of the subject.

24. The method of claim 23 where received real biological data signature is compared with the generated predicted biological data signature to identify at least one biological pathway that is useful for predicting at least one of: age, sex, tissue types, cell types, ethnicity, life expectancy, and combinations thereof.

25. The method of claim 24, where the machine learning platform predicts a biological age, sex, tissue types, cell types, ethnicity, life expectancy or combinations thereof of the synthetic biological data.

26. The method of claim 1, further comprising:
performing a biological signal activation analysis with the synthetic biological data; and
determining a health status of the subject.

27. The method of claim 26, wherein the health status of the subject is an aging rate of the subject.

28. The method of claim 27, further comprising tracking the aging rate of the subject over a time period.

29. The method of claim 26, wherein the health status is a predicted future health status of the subject.

30. The method of claim 29, further comprising identifying a therapeutic protocol to improve the predicted future health of the subject.

31. A computer program product comprising a tangible, non-transitory computer readable medium having a computer readable program code stored thereon, the code being executable by a processor to perform the method of claim 1.

32. The computer program product of claim 31, further comprising:
creating at least a second biological data signature by repeating any one or more of steps (a), (b), (c), and/or (d), wherein the second biological data signature is based on a second real biological data signature from the biological sample of the subject, a different biological sample of the subject, or a second biological sample of a second subject; and
optionally, preparing a report that includes a second synthetic biological data of the second biological data signature.

33. The computer program product of claim 31, further comprising:
comparing the predicted biological data signature with the real biological data signature of the subject;
determining a difference between the synthetic biological data of the subject with the real biological sample of the subject; and
preparing the report with that identifies difference between the synthetic biological data with the real biological sample of the subject.

34. The computer program product of claim 33, further comprising identification of at least one biomarker having a difference between the synthetic biological data with the real biological sample of the subject.

35. The method of claim 34, further comprising identifying at least one biological target, wherein modulation of the at least one biological target modulates the identified at least one biomarker.

36. The computer program product of claim 31, wherein the real biological data signature is based on biological pathway activation signatures for genomics, transcriptomics, proteomics, metabolomics, lipidomics, glycomics, methylomics, or secretomics, and the predicted biological data corresponds with the biological activation signature.

37. The computer program product of claim 36, further comprising at least one of:
correlating a genomics profile with the predicted biological data signature of the subject;
correlating a proteomics profile with the predicted biological data signature of the subject;
correlating a transcriptomics profile with the predicted biological data signature of the subject;
correlating a metabolomics profile with the predicted biological data signature of the subject;
correlating a lipidomics profile with the predicted biological data signature of the subject;
correlating a glycomics profile with the predicted biological data signature of the subject;
correlating a secretomics profile with the predicted biological data signature of the subject; or
correlating a methylomics profile with the predicted biological data signature of the subject.

38. The computer program product of claim 31, further comprising:
performing feature importance analysis for ranking biological data by importance in age prediction by using the real biological data signature; and
identifying a subset biological markers of the biological pathway activation signature thereof that are selected as indicators of a condition of the subject.

39. The computer program product of claim 38, further comprising identifying at least one biological target associated with the condition, wherein modulation of the at least one biological target modulates at least one biomarker of the identified subset of biological markers.

40. The computer program product of claim 31, further comprising correlating the predicted biological data signature with a predicted biological age of the subject.

41. The computer program product of claim 31, further comprising obtaining the real biological data signature by performing a measurement of the genomics, transcriptomics, proteomics, metabolomics, lipidomics, glycomics, methylomics, or secretomics.

42. The computer program product of claim 31, wherein the predicted biological data signature is based on a simulation by a computer program for biological pathway activation signatures for genomics, transcriptomics, proteomics, metabolomics, lipidomics, glycomics, methylomics, or secretomics.

43. The computer program product of claim 31, further comprising conditioning latent codes of the input vectors in a latent space of the machine learning platform with at least one constraint of an attribute of the subject, such that the predicted biological data signature is based on the at least one constraint.

44. The computer program product of claim 43, wherein the synthetic biological data is for a defined biological age of the subject, wherein the predicted biological data signature represents a biological data signature of the subject at the defined biological age.

45. The computer program product of claim 31, wherein the synthetic biological data is for one of:
an aging simulation to increase a biological age of the biological data signature of the subject; or
a rejuvenation simulation to decrease a biological age of the biological data signature of the subject.

46. The computer program product of claim 45, further comprising identification of at least one biomarker having a difference between the real biological sample of the subject with the biological data signature of the aging simulation or the rejuvenation simulation.

47. The computer program product of claim 46, further comprising identifying at least one biological target, wherein modulation of the at least one biological target modulates the identified at least one biomarker.

48. The computer program product of claim 31, after a defined time period,
performing steps (a), (b), (c), (d), and (e) in a second iteration;
comparing the initial report with the report of the second iteration; and
determining a change in the predicted biological data signature over the defined time period.

49. The computer program product of claim 48, further comprising identification of at least one biomarker having a difference over the defined time period.

50. The computer program product of claim 49, further comprising identifying at least one biological target, wherein modulation of the at least one biological target modulates the identified at least one biomarker.

51. The computer program product of claim 48, further comprising:
determining an aging rate over the defined time period based on the change in the predicted biological data signature; and
tracking the change in the predicted biological data signature over the defined time period.

52. The computer program product of claim 31, further comprising:
performing a biological signal activation analysis with the synthetic biological data; and
determining a health status of the subject.

53. The computer program product of claim 52, wherein the health status of the subject is an aging rate of the subject.

54. The computer program product of claim 53, further comprising tracking the aging rate of the subject over a time period.
